(19) United States
(12) Patent Application Publication
(10) Pub. No.: US 2025/0066461 A1
(43) Pub. Date: Feb. 27, 2025

(54) COMPOSITIONS AND METHODS FOR TREATING TRANSPLANT ASSOCIATED THROMBOTIC MICROANGIOPATHY IN BLEEDING PATIENTS

(71) Applicant: Children's Hospital Medical Center, Cincinnati, OH (US)

(72) Inventors: Sonata Jodele, Hawthorne, CA (US); Kana Mizuno, Mason, OH (US); Alexander A. Vinks, Cincinnati, OH (US)

(21) Appl. No.: 18/722,118

(22) PCT Filed: Dec. 21, 2022
(86) PCT No.: PCT/US2022/053603

§ 371 Date: Jun. 20, 2024

Related U.S. Application Data
(60) Provisional application No. 63/292,188, filed on Dec. 21, 2021.

(51) Int. Cl. C07K 16/18; A61K 39/00; A61P 7/02; A61P 37/06; G16H 20/17

(52) U.S. Cl. CPC C07K 16/18; A61P 7/02; A61P 37/06; G16H 20/17; A61K 2039/505; A61K 2039/54; A61K 2039/545

(57) ABSTRACT

The instant disclosure relates to methods for the treatment of an individual having TA-TMA, In one aspect, the methods encompass administration of a C5 inhibitor, more particularly eculizumab, or an antigen-binding fragment thereof. The disclosed methods, in one aspect, may be used for the treatment of individuals having TA-TMA and clinically significant bleeding.

### CROSS-REFERENCE TO RELATED APPLICATIONS

This application claims priority to and benefit of U.S. Provisional Application Ser. No. 63/292,188, filed on Dec. 21, 2021, entitled Eculizumab Precision Dosing Algorithm for Thrombotic Microangiopathy in Children and Young Adults Undergoing HSCT. The contents of each are incorporated in their entirety for all purposes.

### BACKGROUND

Transplant-associated thrombotic microangiopathy (TA-TMA) is a fatal post-transplant complication of hematopoietic stem cell transplantation (HSCT). Untreated patients have very high mortality (16.7% 1 year post HSCT and 9% overall survival).

Survival for TA-TMA has been improved by early intervention with eculizumab, a complement C5 inhibitor, guided by pharmacokinetic/pharmacodynamic (PK/PD) model-informed precision dosing. However, not all patients respond to complement C5 inhibition in the same manner and may not be effectively treated using standard dosing recommendations. As such, targeted therapy, particularly for patients that may be suffering from concurrent blood loss, is needed to more effectively treat TA-TMA patients. The instant disclosure addresses seeks to address one or more of the aforementioned needs in the art.

### BRIEF SUMMARY

The instant disclosure relates to methods for the treatment of an individual having TA-TMA, in particular, via administration of a C5 inhibitor, more particularly eculizumab or a fragment thereof. The disclosed methods, in one aspect, may be used for the treatment of individuals determined to have clinically significant bleeding.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 depicts Eculizumab concentration-time profiles in TA-TMA patients with or without bleeding complications. The Y-axis shows eculizumab observed concentrations and the X-axis shows time after each dose. The black circles represent the data for non-bleeding patients (n=38) and the red circles represent the data for bleeding patients (n=19). The data collected from the same patients are connected by dotted lines.

FIG. 2 depicts Eculizumab clearance and sC5b-9 change over the doses of therapy. PK and PD changes over treatment doses were evaluated using individual eculizumab clearance estimates (A) and pre-dose sC5b-9 levels (B). The red and black circles represent the data in bleeding patients and non-bleeding patients respectively. Eculizumab individual clearance values at each week were estimated by Bayesian estimation method with consideration of inter-occasion variability and adjusted by allometrically scaled bodyweight of 70 kg. As the study included a wide age range (0.5-29.9 years), the effect of body size increase on eculizumab clearance was taken into account by adjusting it by allometric scaling to a bodyweight of 70 kg to allow comparison across pediatric and young adult patients with different body sizes as previously described.7 Eculizumab clearance estimates and sC5b-9 level changes over time were statistically analyzed using one-way ANOVA (R 3.0.3).

FIG. 3 depicts a simulation of eculizumab concentration-time profiles. Eculizumab concentration-time profiles over 7 days after the first dose were simulated based on our developed models for non-bleeding and bleeding patients shown in Table 2. The population PK parameter estimates were used for the simulations. Eculizumab concentration-time curves are shown in different colors and represent different pre-dose sC5b-9 levels ranging from 200 to 800 ng/mL. The horizontal dotted pink line represents the suggested eculizumab target concentration of 100 μg/mL.

FIG. 4 depicts a Simulation for optimal dosing algorithms for non-bleeding patients with TA-TMA. The y-axis shows the probability of target attainment determined as the proportion of patients who achieved eculizumab concentrations above the target, and the y-axis shows each bodyweight cohort. A total of 12,000 age-bodyweight-matched subjects was randomly sampled from the CDC-NHANES database. Six bodyweight cohorts were defined as follows: <10 kg, 10-<20 kg, 20-<30 kg, 30-<40 kg, 40-<70, and 70-<100 kg. Realistic pre-dose sC5b-9 levels were generated using simulation to be matched to the observed sC5b-9 distribution. A Monte Carlo Simulation analysis was conducted to predict eculizumab trough concentrations for each dosing scenario using NONMEM.

FIG. 5 depicts the probability of target attainment for various dosing schedules. The x-axis shows eculizumab dosing intervals ranged from 1 to 7 days. The y-axis shows the probability of target attainment % to reach eculizumab target trough concentration ≥100 g/mL. The probability of target attainment was predicted for dosing protocols with different dose amounts (mg) ranging from 300 to 2100 mg and are shown in lines with different colors. PTA % close to 100% could be achieved by increasing the dose by 300 mg (one vial) in each bodyweight cohort of protocol 4 shown in FIG. 4.

FIG. 6A-6D depict the population PK model evaluation for final models. Goodness-of-fit plots (A and C) and prediction-corrected visual predictive check (pcVPC) (B and D) for the final population PK model for non-bleeding and bleeding patients shown in Table 2. Open circles represent observations. A, C: Goodness-of-fit plots includes four figures below: observed eculizumab concentrations vs. population model predicted concentrations, observed concentrations vs individual predicted concentration estimated using Bayesian estimation method, conditional weighted residuals vs population predicted concentrations, and conditional weighted residuals vs time after the dose. B, D: The red lines show 5th (dashed line), 50th (solid line) and 95th (dashed line) percentiles of observed eculizumab concentrations. The shaded areas show 5th (blue), 50th (red) and 95th (blue) percentiles for simulated data based on the final model. Goodness-of-fit plots showed the model stability of the final model. The prediction corrected-visual predictive check (pcVPC) showed that the median, and the 5th and 95th percentile prediction intervals were in good agreement with the observations.

FIG. 7A-7B depict schematics showing the number of red blood cell (RBC) (7A) and platelet transfusion (7B) during the first 5 doses of therapy in bleeding and non-bleeding patients. The y-axis shows the sum of each platelet transfusion (mg/kg/day) during each dosing cycle. Each transfusion during each dosing interval was shown in a different color. 15-16 out of 38 bleeding patients received transfusions, whereas only 2 out of 38 non-bleeding patients received transfusions after starting eculizumab therapy. The maximum number of transfusions for RBC and platelet were 12 and 32, respectively. Bleeding patients received significantly higher transfusion compared to non-bleeding patients across all first 5 doses of therapy.

FIG. 8 depicts graphs showing the effect of red blood cell (RBC) transfusion on eculizumab clearance in bleeding patients. The x axis shows the total amount of red blood cell transfusion during dosing intervals. Since body weight and pre-treatment sC5b-9 were significant covariates on eculizumab clearance, the eculizumab clearance on the y-axis was adjusted by allometrically scaled body weight of 70 kg and pretreatment sC5b-9 of 244 ng/mL. The linear regression analysis showed weak correlation between eculizumab clearance and RBC transfusion (R2=0.13, p<0.01) or platelet transfusion (R2=0.20, p<0.01). The correlation between RBC and platelet transfusion was observed.

FIG. 9 is a graph depicting the probability of target attainment for protocol 4 with consideration of pre-dose sC5b-9 levels. The probability of target attainment for protocol 4 shown in FIG. 5 was stratified by different sC5b-9 cohorts (<250, 250-<500, 500-<750 and >750 ng/mL) and are shown in different colors. The x-axis shows different body weight cohorts, and the y-axis shows the probability of target attainment to reach eculizumab trough concentrations ≥100 μg/mL.

FIG. 10. Selection of the duration of the loading dose for TA-TMA patients with active GI bleeding. Panel A depicts sC5b-9 changes over the first 3 weeks of eculizumab therapy. The blue circles with lines represent the measured sC5b-9 levels over the first 3 weeks of eculizumab therapy for nonbleeding patients (top panel), and the magenta represents those for bleeding patients (bottom panel). The data were stratified by the pretreatment sC5b9 levels of below normal <244 ng/mL (left panel), intermediate levels above the target but below the doubled normal (244-<488 ng/mL, middle panel), or higher ≥488 ng/mL (right panel). Each connected line shows the data from an individual patient. Due to a limited number of patients who had extremely high sC5b-9 at the start of therapy (bottom right), a significant sC5b-9 reduction delay observed in non-bleeding patients (top right) was not observed for bleeding patients (bottom right). However, considering comparable overall sC5b-9 distribution regardless of the bleeding, the loading dose for active bleeding was proposed at least for the first 14 days as proposed for non-bleeding patients by taking worst-case scenarios. Panel B depicts Frequencies of patients who achieved sC5b-9 levels <244 ng/mL over the first 3 weeks of therapy. The blue represents the data for nonbleeding, and the magenta represents the data for bleeding patients. The frequencies of patients who achieved sC5b-9 below the normal <244 ng/mL were calculated by dividing the patient count achieving the normal by the total count of patients who had measured sC5b-9 levels available each day. 20% of the bleeding patients showed sC5b-9<244 ng/mL at the start of therapy which increased over time up to 90% by 14 days of the eculizumab therapy. These suggest that the loading dose of the first 14 days of therapy can give benefits to bleeding patients.

FIG. 11. Loading dose protocol for non-bleeding patients predicts lower target attainment for bleeding patients. Option A (Q72h dosing option) proposed for non-bleeding patients predicts a significantly lower probability of target attainment in bleeding patients than in non-bleeding patients, especially with higher body weight as shown in the arrows. In Option B (more flexible dosing interval option) for non-bleeding patients, the probability of target attainment reaches 80% or more in bleeding patients except for patients 40-<70 kg. For TA-TMA patients with lower body weight <20 kg, the dosing protocol for non-bleeding patients can be proposed to those who have active GI bleeding.

FIG. 12. Optimal “Loading Dose” Simulations of eculizumab for TA-TMA with active GI bleeding. The probability of target attainment was simulated for the selection of “loading” mg doses and dosing intervals based on population PK model developed for bleeding patients assuming the similar sC5b-9 distribution as observed in bleeding patients of Applicant's published data (Mizuno et al 2022). The simulation was performed with a variety of mg dose amounts ranging from 300 mg to 2100 mg with the dosing intervals ranging from 1-7 days. Different mg dose amounts are shown. The optimal dosing options were selected to achieve the probability of target attainment of 80% or more. The optimal loading dose protocols with dashed lines were selected and are summarized in Tables 1 and 3.

FIG. 13. Selection of Optimal “Induction Dosing” of eculizumab for TA-TMA with active gastrointestinal (GI) bleeding. Optimal dosing simulations were conducted by simulating eculizumab C_{trough} using the Monte Carlo Simulation approach based on the published population PK/PD model (Mizuno et al, Blood Adv 2022). The y-axis shows the simulated C_{trough} of eculizumab, and the x-axis shows the body weight cohorts. Different eculizumab mg dose amounts from 300 mg to 1500 mg at 300 mg (1 vial) increments are shown. When bleeding patients receive the proposed loading doses of eculizumab proposed for active bleeding during the induction phase, significantly high C_{trough} levels were achieved (left panel). The lower interquartile C_{trough} falls to the target level by spacing out by 1-2 days with the same dose amount except for patients with the highest body weight over 70 kg. The highest body weight group requires additional 1 vial (300 mg) to achieve the lower interquartile C_{trough} levels above the target level of 100 μg/mL. The optimized protocol shown in the right panel was summarized in the full dosing protocol in Table 1.

FIG. 14. Optimal “Induction Dose” Simulations of eculizumab for TA-TMA with active GI bleeding to explore additional dosing options. PK simulations were conducted using the same method above in FIG. 12, but with sC5b-9 distribution observed for the “induction” dose phase instead of the “loading” dose phase. Additional potential dosing protocols were proposed as shown in dashed lines and are summarized in Tables S3(B) as induction dosing options.

### DETAILED DESCRIPTION

### Definitions

Unless otherwise noted, terms are to be understood according to conventional usage by those of ordinary skill in the relevant art. In case of conflict, the present document, including definitions, will control. Exemplary methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “antibody fragment,” “antigen-binding fragment,” or similar terms refer to fragment of an antibody that retains the ability to bind to an antigen (e.g., a complement component C5 protein), e.g., a single chain antibody, a single chain Fv fragment (scFv), an Fd fragment, an Fab fragment, an Fab′ fragment, or an F(ab′)2 fragment. An scFv fragment is a single polypeptide chain that includes both the heavy and light chain variable regions of the antibody from which the scFv is derived. In addition, diabodies and intrabodies that bind to a complement component C5 protein can be incorporated into the compositions, and used in the methods, described herein.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

The methods may comprise, consist of, or consist essentially of the elements of the methods as described herein, as well as any additional or optional element described herein or otherwise useful in methods of treating an individual with transplant-associated thrombotic microangiopathy (TA-TMA) due to hematopoietic stem cell transplantation (HSCT) and a bleeding complication.

Patients with TA-TMA and clinically significant intestinal bleeding require personalized eculizumab dosing. In certain clinical situations real time drug PK values are not readily available. Disclosed are “fixed” dosing regimens, or dosing tables, for TA-TMA therapy using eculizumab for patients with gastrointestinal bleeding based. The instant disclosure relates to complement C5 inhibitor, in particular eculizumab, pharmacokinetics/pharmacokinetics (PK/PD), and dosing regimens for complement inhibitor. Eculizumab is an C5 inhibitor sold under the tradename Soliris®, manufactured by Alexion Pharmaceuticals, Inc., Boston, MA and is described in Kaplan (2002) Curr Opin Investig Drugs 3(7):1017-23; Hill (2005) Clin Adv Hematol Oncol 3 (11): 849-50; and Rother et al. (2007) Nature Biotechnology 25(11): 1256-1488), in particular, administration of eculizumab to an individual having TA-TMA, who may further be diagnosed as having clinically significant bleeding. The disclosed methods may be used to treat both TA-TMA patients having active gastrointestinal bleeding, and/or TA-TMA patients having resolved gastrointestinal bleeding. For example, the methods disclosed herein encompass the dosing schedule as set forth in Table 1.

In one aspect, a method of treating an individual with transplant-associated thrombotic microangiopathy (TA-TMA) and active gastrointestinal bleeding is disclosed. In this aspect, the method may comprise administering eculizumab, or antigen binding fragment thereof, to said individual wherein the eculizumab or antigen binding fragment thereof is administered via a loading dose and an induction dose. In one aspect, the loading dose may comprise about 1200 milligrams every 24 hours for four doses, followed by about 1500 milligrams every 48 hours for five doses, wherein said individual is 70 kilograms or more, or about 900 milligrams every 24 hours for four doses followed by about 900 milligrams every 48 hours for five doses, wherein said individual has a weight of 40 kilograms to less than 70 kilograms, or about 900 milligrams every 48 hours for two doses followed by three doses every 72 hours, wherein said individual has a weight of 30 kilograms to less than 40 kilograms, or about 900 milligrams every 48 hours for two doses followed by about 900 milligrams every 72 hours for three doses, wherein said individual has a weight of 20 kilograms to less than 30 kilograms, or about 600 milligrams every 72 hours for five doses, wherein said individual has a weight of 10 kilograms to less than 20 kilograms or about 300 milligrams every 72 hours for five doses, wherein said individual has a weight of less than 10 kilograms. The induction dose may comprise about 1500 milligrams every 48 hours for 3 weeks, wherein said individual has a weight of 70 kilograms or more, or about 900 milligrams every 48 hours for 3 weeks, wherein said individual has a weight of 40 kilograms to less than 70 kilograms, or about 900 milligrams every 72 hours for 3 weeks, wherein said individual has a weight of 30 kilograms to less than 40 kilograms, or about 900 milligrams every 96 hours for 3 weeks, wherein said individual has a weight of 20 kilograms to less than 30 kilograms for 3 weeks, or about 600 milligrams every 96 hours for 3 weeks, wherein said individual has a weight of 10 kilograms to less than 20 kilograms, or about 300 milligrams every 96 hours for 3 weeks, wherein said individual has a weight of less than 10 kilograms.

In one aspect, a method of treating an individual with transplant-associated thrombotic microangiopathy (TA-TMA) and resolved gastrointestinal bleeding is disclosed. In this aspect, the method may comprise administering eculizumab, or antigen binding fragment thereof, to said individual wherein the eculizumab or antigen binding fragment thereof is administered via an induction dose and a maintenance dose. In one aspect, the induction dose may comprise about 1500 milligrams twice weekly every three to four days for four weeks, wherein said individual has a weight of 70 kilograms or more, or about 1500 milligrams weekly for four weeks, wherein said individual has a weight of 40 kilograms to less than 70 kilograms, or about 1200 milligrams weekly for four weeks, wherein said individual has a weight of 30 kilograms to less than 40 kilograms, or about 900 milligrams weekly for four weeks, wherein said individual has a weight of 20 kilograms to less than 30 kilograms, or about 600 milligrams weekly for four weeks, wherein said individual has a weight of 10 kilograms to less than 20 kilograms, or about 300 milligrams weekly for four weeks, wherein said individual has a weight of less than 10 kilograms. The maintenance dose may comprise about 1200 milligrams twice weekly every three to four days for four to five weeks, wherein said individual is 70 kilograms or more, or about 1200 milligrams weekly for four to five weeks, wherein said individual has a weight of 40 kilograms to less than 70 kilograms, or about 900 milligrams weekly for four to five weeks, wherein said individual has a weight of 30 kilograms to less than 40 kilograms, or about 600 milligrams weekly for four to five weeks, wherein said individual has a weight of 20 kilograms to less than 30 kilograms, or about 600 mg weekly for four to five weeks, wherein said individual has a weight of 10 kilograms to less than 20 kilograms, or about 300 milligrams every two weeks for four to five weeks, wherein said individual has a weight of less than 10 kilograms.

In a further aspect, a method of treating an individual with transplant-associated thrombotic microangiopathy (TA-TMA), comprising administering eculizumab, or antigen binding fragment thereof, to said individual is disclosed, wherein said eculizumab or antigen binding fragment thereof is administered at a dose of about 300 milligrams to a patient weighing <10 kilograms, or about 600 milligrams to a patient weighing 10 kilograms to <20 kilograms, or about 900 milligrams to a patient weighing 20 to <30 kilograms, or about 1200 milligrams to a patient weighing 30 to <40 kilograms, or about 1500 milligrams to a patient weighing 40 to <70 kilograms, or 2100 milligrams to a patient weighing 70 to <100 kilograms, said dose being administered every three days, wherein said patient is a non-bleeding patient.

In a further aspect, a method of treating an individual with TA-TMA comprising administering to eculizumab, or antigen binding fragment thereof to said individual, is disclosed wherein said eculizumab or antigen binding fragment thereof is administered at a dose of about 300 milligrams every three days to a patient weighing <10 kilograms, or about 600 milligrams every three days to a patient weighing 10 kilograms to <20 kilograms, or about 600 milligrams every two days to a patient weighing 20 to <30 kilograms, or about 600 milligrams a day to a patient weighing 30 to <40 kilograms, or about 900 milligrams a day to a patient weighing 40 to <70 kilograms, or about 900 milligrams a day to a patient weighing 70 to <100 kilograms.

In a further aspect, a method of treating an individual with TA-TMA, the method comprising administering eculizumab, or antigen binding fragment thereof to said individual, is disclosed, wherein said eculizumab or antigen binding fragment thereof is administered at a dose of about 300 milligrams every three days to a patient weighing <10 kilograms, or about 600 milligrams every three days to a patient weighing 10 kilograms to <20 kilograms, or about 900 milligrams every three days to a patient weighing 20 to <30 kilograms, or about 900 milligrams every two days to a patient weighing 30 to <40 kilograms, or about 1200 milligrams every two days to a patient weighing 40 to <70 kilograms, or about 1200 milligrams a day to a patient weighing 70 to <100 kilograms.

In a further aspect, a method of treating an individual with TA-TMA is disclosed, the method comprising administering eculizumab, or antigen binding fragment thereof, to said individual wherein said eculizumab or antigen binding fragment thereof is administered at a dose of about 40 milligrams/kilograms every three days to a patient weighing <10 kilograms, or 30 milligrams/kilograms every two days to a patient weighing 10 kilograms to <100 kilograms.

In one aspect, the individual having TA-TMA has high-risk TMA with MODS. In one aspect, the TA-TMA may be characterized by a sC5b-9 level that is at least twice the measured baseline level in said individual, and/or by a sC5b-9 level that is greater than about 244 nanograms per milliliter.

The administration of any of the foregoing treatment regimens (dosing schedules) may be initiated at the time of TA-TMA diagnosis in said individual. In one aspect, the administration may be via intravenous administration. The methods may be carried out until a hematological TA-TMA response selected from one or more of normalization of LDH, resolution of the need for red blood cell (RBC) and platelet transfusions, and disappearance of schistocytes is achieved in the individual being treated. In one aspect, the induction dose may be carried out until a hematological TA-TMA response of normalization of LDH, resolution of the need for red blood cell (RBC) and platelet transfusions, and disappearance of schistocytes is achieved in the individual being treated. In a further aspect, the induction dose may be carried out until a normalized sC5b-9 is achieved, the normalized sC5b-9 being one or both of an sC5b-9 level of less than 244 ng/mL or an sC5b-9 level that is substantially at baseline (pre-transplant), or until an elevated sC5b-9 level is normalized.

In certain aspects, the methods may comprise determining whether an individual has clinically significant gastrointestinal bleeding prior to selecting the appropriate dosing regimen. In one aspect, the individual may be determined to have resolved gastrointestinal bleeding. In certain aspects, the individual to be treated using the aforementioned dosing regimens may not have clinically significant bleeding (i.e., a “non-bleeding status” individual). In some aspects, the individual may be selected from an infant, a pre-pubescent individual, or an adult (greater than 18 years of age).

In a further aspect, a precision dosing tool for determining a course of treatment in an individual diagnosed with TA-TMA is disclosed. In this aspect, the tool may employ the method of detecting one or more patient-specific variables, and determining a therapeutically effective amount of an eculizumab therapy, wherein said determining employs an algorithm selected from

$$CL = {CL}_{L} + {CL}_{NL} , \\ {CL}_{L} = {CL}_{L , pop} \times {( WT / 70 )}^{0.97} , \\ {CL}_{NL} = {CL}_{NL , pop} \times {( sC 5 b - 9 / 244 )}^{0.53} \times {( WT / 70 )}^{0.97} , \\ Vd = {Vd}_{pop} \times {( WT / 70 )}^{0.63} ( for non - bleeding patients ) ;$$

$$and$$

$$CL = {CL}_{L} + {CL}_{NL} , \\ {CL}_{L} = {CL}_{L , pop} \times {( WT / 70 )}^{1.03} , \\ {CL}_{NL} = {CL}_{NL , pop} \times {( sC 5 b - 9 / 244 )}^{0.52} \times {( WT / 70 )}^{1.03} , \\ Vd = {Vd}_{pop} \times {( WT / 70 )}^{0.74} ( for bleeding patients ) ;$$

wherein CL_{NL}, is the eculizumab population mean nonlinear clearance for a 70 kg-patient which represents a target-mediated component of clearance, CL_{L,pop}, is the eculizumab population mean linear clearance for a 70 kg-patient which represents a nonspecific component of clearance mediated by the neonatal Fc receptor, C_{tot,pop} is the population mean total clearance defined as sum of CL_{NL,pop} and CL_{L,pop}, Vdₚₒₚ, is the population mean volume of distribution for a 70 kg-patient, and WT is actual body weight (kg); and administering a therapeutically effective amount of eculizumab to an individual as described herein, according to said algorithm. Such methods may be carried out using a computerized device, for example, a handheld device that may be used “bedside”, at point of care.

The disclosed methods may comprise one or more of a loading dose, an induction dose, and a maintenance dose.

LOADING DOSE. In one aspect, the loading dose may be used to control complement activation by achieving normalization of sC5b-9. An intensive loading dose may be used to normalize sC5b-9 as fast as possible by providing adequate eculizumab dose to fully suppress complement activity in blood. The loading dose may use the maximum doses (mg) for patient weight, as TA-TMA clinical response is unlikely to be achieved unless sC5b-9 is normalized. In one aspect, the loading dose is carried out until normalize sC5b-9 is normalized. In one aspect, eculizumab may be administered effective to normalize sC5b-9 within a two week (14 day) period. In one aspect, eculizumab may be administered to a patient having elevated sC5b-9 for a period of about 11 to about 13 days. Elevated sC5b-9 may be one or both of >244 ng/ml or doubled as compared to the patient's baseline value.

In one aspect, the disclosed methods may employ a 72 hour interval dosing schedule, or a 48 hour dosing schedule, or a 24 hour dosing schedule. In one aspect, each dose (mg) is given at no longer than 72 hour intervals in subjects with elevated sC5b-9. In one aspect, the loading dose may be multiple doses, which may be administered in a loading dose interval, which may be at least about, or about two weeks.

INDUCTION DOSE. The induction dose may be used to control sC5b-9 activation while achieving clinical TA-TMA response. In one aspect, the induction dosing time is for at least about three weeks, or at least about four weeks, or about four weeks. In one aspect, the induction dose may be three weeks, but can be shortened or extended based on the bleeding status of the patient treated. In one aspect, the total therapy time with loading and induction is at least about six weeks, wherein the loading dose comprises about two weeks of the total loading dose and induction dose therapy period. In one aspect, the sC5b-9 activation is controlled with the loading dose period, such that when the induction dosing is started, the sC5b-9 level is normalized (less than about 244 ng/mL or substantially returned to patient baseline) when the induction dosing is started. In one aspect, the induction period is administered to a patient having a normalized sC5b-9 level, and is administered every seven days, or every six days, or every five days, or every four days, or every three days, or every two days, or every day, or twice a day. In one aspect, the induction dose is administered for no more than seven days. In one aspect, the patient has a weight of ≥70 kg, and is administered twice a week dosing during the induction period.

MAINTENANCE DOSING. Eculizumab clearance in patients with TA-TMA was still higher by 23% in maintenance phase as compared to those with aHUS. The maintenance dosing period may be used to sustain TA-TMA control and normalized sC5b-9 levels. During the maintenance dosing period, complement activation may be assessed. In one aspect, when complement activation is resolved, eculizumab therapy can be safely stopped. In one aspect, the maintenance dosing phase is at least, or about 4 weeks. Applicant thus proposed a weekly drug dosing option for all weight groups except for those >70 kg that required twice weekly dosing and those <10 kg who only required dosing every 2 weeks using a weight-based drug (mg) dose to sustain a therapeutic eculizumab drug level of ≥75 μg/ml resulting in desired clinical response.

In one aspect, the dosing schedule comprises a total dosing time of about 9.5 weeks, with about 5 to about 6 weeks of loading and induction therapy, and about 4 to about 5 of maintenance therapy. In one aspect, if, after four weeks of maintenance therapy, sC5b-9 remains normalized and eculizumab drug concentration (trough) is >100 μg/ml and/or is rising with each dose, then therapy can be discontinued.

In some aspects, the patient has not previously been treated with a complement inhibitor (e.g., the patient is a complement inhibitor treatment-naïve patient). In some aspects, the patient may have been previously treated with a complement blocker, but may have cleared such previous treatment.

In some aspects, the individual is an infant. The individual may be, e.g., 0.5 (e.g., 1, 1.5, 2, 2.5, 3, 3.5, 4, 4.5, 5, 5.5, 6, 6.5, 7, 7.5, 8, 8.5, 9, or 9.5) years old. The infant can be less than 10 (e.g., less than 9.5, 9, 8.5, 8, 7.5, 7, 6.5, 6, 5.5, 5, 4.5, 4, 3.5, 3, 2.5, 2, 1.5, 1, or less than 1) year(s) old. In some aspects, the individual is an infant. The individual may be, e.g., 0.5 (e.g., 1, 1.5, 2, 2.5, 3, 3.5, 4, 4.5, 5, 5.5, 6, 6.5, 7, 7.5, 8, 8.5, 9, or 9.5) years old. The infant can be less than 10 (e.g., less than 9.5, 9, 8.5, 8, 7.5, 7, 6.5, 6, 5.5, 5, 4.5, 4, 3.5, 3, 2.5, 2, 1.5, 1, or less than 1) year(s) old. In one aspect, the individual is a neonate (from birth through the first 28 days of life), or an infant (29 days of age to less than 2 years of age), a child (2 years of age to less than 12 years of age) or an adolescent (aged 12 through 21, up to but not including the 22nd birthday).

In one aspect, a method of treating an individual having TA-TMA is disclosed, wherein and individual is administered a C5 inhibitor, or eculizumab, or a fragment thereof, according to the following dosing table:

TABLE 1
Eculizumab full dosing regimen for TA-TMA
patients with GI bleeding.

| | | | GI bleed ACTIVE | |
|---|---|---|---|---|
| | GI bleed RESOLVED | | (after GI bleeding is resolved) | |
| | | Induction | Induction | |
| Bleeding | Loading dose | dose-1^{*2} | dose-2^{*3} | Maintenance^{*3} |
| status^{*1} | (2 weeks) | (3 weeks) | (4 weeks) | (4-5 weeks) |
| 70 kg or | 1200 mg IV Q24 h × 4 | 1500 mg IV Q48 h | 1500 mg IV twice | 1200 mg IV |
| more | doses then 1500 mg | | weekly (Q3-4d) | twice weekly |
| | Q48 h × 5 doses | | | (Q3-4d) |
| 40 kg to | 900 mg IV Q24 h × 4 | 900 mg IV Q48 h | 1500 mg IV weekly | 1200 mg IV |
| <70 kg | doses then Q48 h × 5 | | | weekly |
| | doses | | | |
| 30 kg to | 900 mg IV Q48 h × 2 | 900 mg IV Q72 h | 1200 mg IV weekly | 900 mg IV |
| <40 kg | doses then Q72 h × 3 | | | weekly |
| | doses | | | |
| 20 kg to | 900 mg IV Q48 h × 2 | 900 mg IV Q96 h | 900 mg IV weekly | 600 mg IV |
| <30 kg | doses then Q72 h × 3 | | | weekly |
| | doses | | | |
| 10 kg to | 600 mg IV Q72 h × 5 | 600 mg IV Q96 h | 600 mg IV weekly | 300 mg IV |
| <20 kg | doses | | | weekly |
| <10 kg | 300 mg IV Q72 h × 5 | 300 mg IV Q96 h | 300 mg IV weekly | 300 mg IV |
| | doses | | | every 2 weeks |

*1: Bleeding status for TA-TMA patients who experience GI bleeding; Active and resolved bleeding phases are expected during the therapy for bleeding patients. The criteria for active gastrointestinal (GI) bleeding were described in the patent document. *2: Loading dose and Induction dose-1: The loading dose and the induction dose-1 were designed for “active” GI bleeding. Considering the continuous high clearance prediction in TA-TMA patients who had “active” bleeding (Mizuno et al 2022), less mg dose amount with more frequent doses than non-bleeding patients shown in Table 2 to maintain the target Ctrough>100 μg/mL. This induction dose was selected from the 2 proposed induction dosing options shown in Table 3 in consideration of financial and pharmacological benefits. The detailed explanations for the selection were described in Table 3. In accordance with the reduction of sC5b-9, the dosing interval for patients with active bleeding can be spaced out but is limited to up to 96 h of intervals for patients<30 kg, 72 h of intervals for patients 30-<40 kg, and 48 h of intervals for patients >=40 kg to achieve target Ctrough>100 μg/mL. Induction dose-1 therapy course is proposed to be 3 weeks based on PK modeling using clinical data in treated patients, but it can be shortened or extended based on bleeding status of the patient treated. *3: Induction dose-2& maintenance dose: The induction dose-2 followed by the maintenance dose is given for patients after the bleeding is resolved. These protocols were developed using non-bleeding patients' data as shown in Table 3.

The dosing regimen of Table 1 may be used for two categories of bleeding patients: active and resolved bleeding. The “loading dose and the induction dose-1” may be used for “active” GI bleeding, and the “induction dose-2 followed by the maintenance dose” may be used for patients after the bleeding is resolved.

The continuously high clearance is predicted in TA-TMA patients who had “active” bleeding (Mizuno et al 2022). Bleeding patients require less mg dose amount with more frequent doses than non-bleeding patients to maintain C_{trough} above the target level. (Table 1 vs Table 2)

TABLE 2
Dosing regimen for TA-TMA for non-bleeding patients

| | Loading dose | Induction dose | Maintenance |
|---|---|---|---|
| Weight | (2 weeks) | (4 weeks) | (4-5 weeks) |
| 70 kg or more | 1200 mg IV Q24 h × 4 | 1500 mg IV twice | 1200 mg IV twice |
| | then Q72 h × 3 doses | weekly (Q3-4d) | weekly |
| 40 kg to <70 kg | 1200 mg IV Q48 h × 2 | 1500 mg IV weekly | 1200 mg IV weekly |
| | then Q72 h × 3 doses | | |
| 30 kg to <40 kg | 900 mg IV Q48 h × 2 | 1200 mg IV weekly | 900 mg IV weekly |
| | then Q72 h × 3 doses | | |
| 20 kg to <30 kg | 600 mg IV Q48 h × 2 | 900 mg IV weekly | 600 mg IV weekly |
| | then Q72 h × 3 doses | | |
| 10 kg to <20 kg | 600 mg IV Q72 h × 5 | 600 mg IV weekly | 300 mg IV weekly |
| | doses | | |
| <10 kg | 300 mg IV Q72 h × 5 doses | 300 mg IV weekly | 300 mg IV every 2 weeks |

A financially effective dosing option was selected as the full dosing regimen for TA-TMA with active GI bleeding. (Table 3-A&B)

TABLE 3A
Dosing options of eculizumab for TA-TMA with GI bleeding
Loading dose options

| | Option A | |
|---|---|---|
| Weight | (More financially effective) | Option B |
| 70 kg or more | 1200 mg IV Q24 h | |
| 40 kg to <70 kg | 900 mg IV Q24 h | |
| 30 kg to <40 kg | 900 mg IV Q48 h | 600 mg IV Q24 h |
| 20 kg to <30 kg | (number of vials: | (number of vials: |
| | 1.5 × days of | 2.0 × days of |
| | eculizumab therapy) | eculizumab therapy) |

| | |
|---|---|
| 10 kg to <20 kg | 600 mg IV Q72 h |
| <10 kg | 300 mg IV Q72 h |

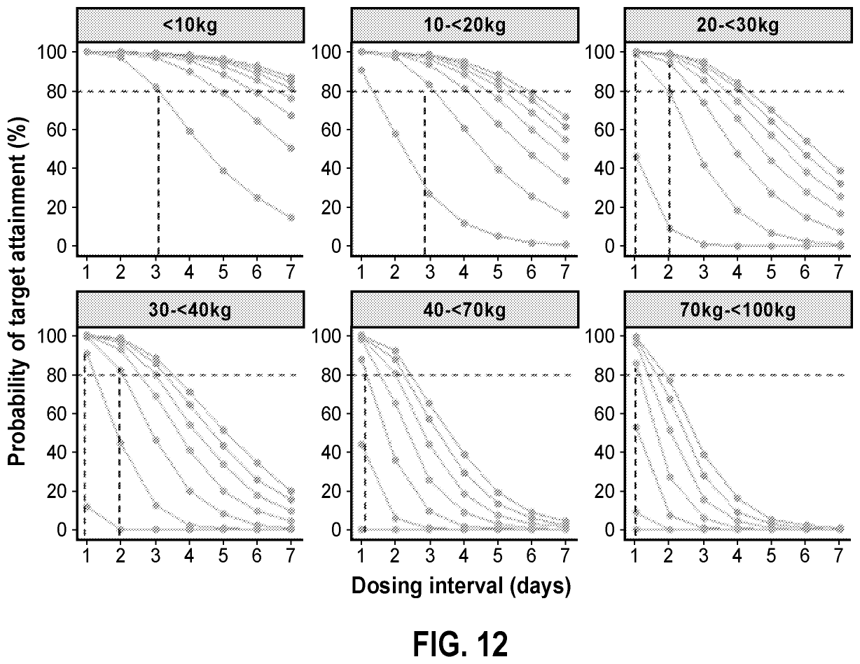

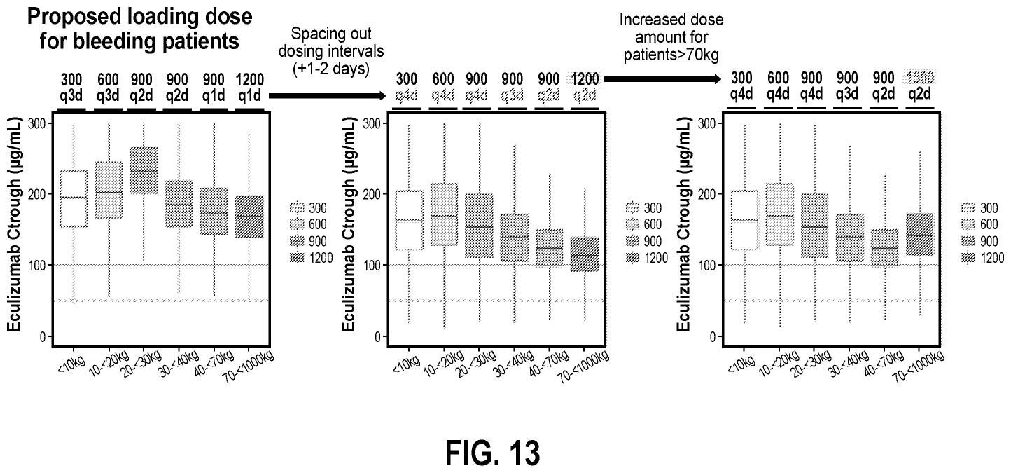

The loading dose options were selected based on the simulations in FIG. 12. Option A was selected for the full dosing proposal in Table 1 in consideration of financial benefits.

### (A) Induction Dosing Options

TABLE 3B
Dosing options of eculizumab for TA-TMA with GI bleeding
Option A (More financially effective)

| Weight | Induction dose (3 weeks)* |
|---|---|
| 70 kg or more | 1500 mg IV Q48 h (number of vials: 2.50 × days of eculizumab therapy)* |
| 40 kg to <70 kg | 900 mg IV Q48 h (number of vials: 1.50 × days of eculizumab therapy) |
| 30 kg to <40 kg | 900 mg IV Q72 h (number of vials: 1.00 × days of eculizumab therapy) |
| 20 kg to <30 kg | 900 mg IV Q96 h (number of vials: 0.75 × days of eculizumab therapy) |
| 10 kg to <20 kg | 600 mg IV Q96 h (number of vials: 0.50 × days of eculizumab therapy) |
| <10 kg | 300 mg IV Q96 h (number of vials: 0.25 × days of eculizumab therapy) |

TABLE 3C
Dosing options of eculizumab for TA-TMA with GI bleeding
Option B (Increased mg dose amount with minimum frequency)

| Weight | Induction dose (3 weeks)** |
|---|---|
| 70 kg or more | 1500 mg IV Q48 h (number of vials: 2.50 × days of eculizumab therapy)* |
| 40 kg to <70 kg | 1500 mg Q72 h (number of vials: 1.67 × days of eculizumab therapy) |
| 30 kg to <40 kg | 1500 mg Q96 h (number of vials: 1.25 × days of eculizumab therapy) |
| 20 kg to <30 kg | 1500 mg Q120 h (number of vials: 1.00 × days of eculizumab therapy) |
| 10 kg to <20 kg | 1200 mg weekly (number of vials: 0.57 × days of eculizumab therapy) |
| <10 kg | 600 mg weekly (number of vials: 0.29 × days of eculizumab therapy) |

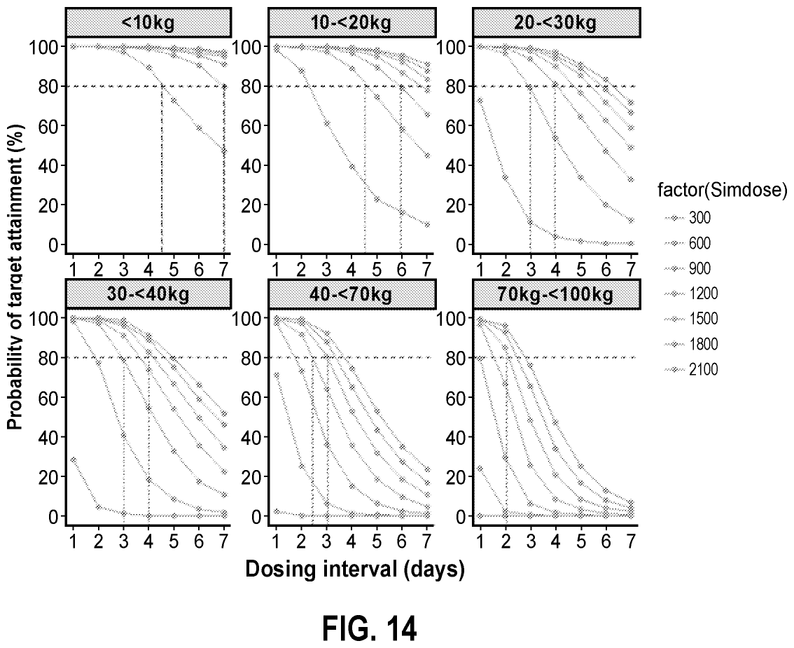

*Only one induction dosing option was suggested for patients with 70 kg or more since a higher dose of 1800 mg or 2100 mg dose with 3 days of dosing intervals does not reach the target attainment of 80%. Option A is designed as dosing using a lower dose of drug in mg, but with more frequent dosing, and option B utilizes an increased dose in mg but with a minimum frequent dosing. Option A may be used as the dosing regimen for patients with active bleeding considering financial and pharmacological benefits. Option A requires fewer vials for the treatment and is financially effective compared to option B. Overall, option A achieves a higher probability of target attainment than option B (FIG. 14). ** Induction dose-1 therapy course is proposed to be 3 weeks based on PK modeling using clinical data in treated patients, but can be shortened or extended based on bleeding status of the patient treated.

In accordance with the reduction of sC5b-9, the dosing interval for patients with active bleeding can be spaced out but is limited to up to 96 h of intervals for patients<30 kg, 72 h of intervals for patients 30-<40 kg, and 48 h of intervals for patients >=40 kg to achieve target Ctrough>100 μg/mL.

Dosing for Patients with Active GI Bleeding

### Loading Dose

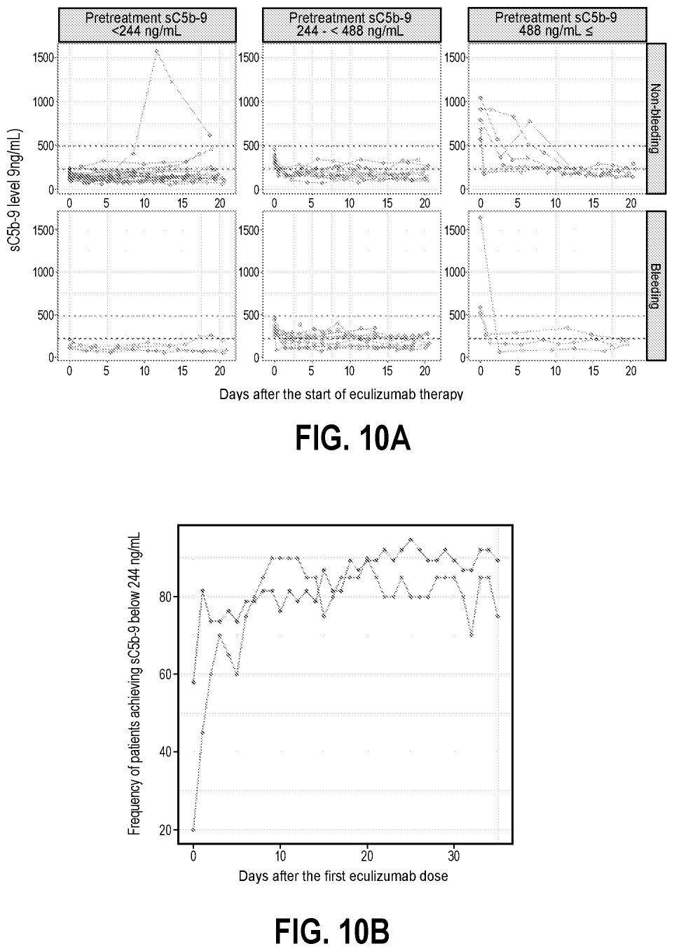

Loading dosing time (2 weeks) (FIG. 10). As described for non-bleeding, normalization of sC5b-9 should occur no longer than within 2 weeks (14 days) as sustained complement activation for longer than two weeks is shown to be associated with multi-organ injury in TA-TMA. Applicant found that in a non-bleeding patient with double normal sC5b-9 it may take from about 11 to about 13 days to normalize sC5b-9 even with adequate (therapeutic eculizumab dose) and fully suppressed total blood complement activity (CH50<10% lower normal limit). A limited number of patients showed double normal sC5b-9 at the start of therapy in bleeding patients. Consideration of comparable sC5b-9 distribution throughout the therapy, the loading dose duration observed for non-bleeding can be applied for bleeding patients.

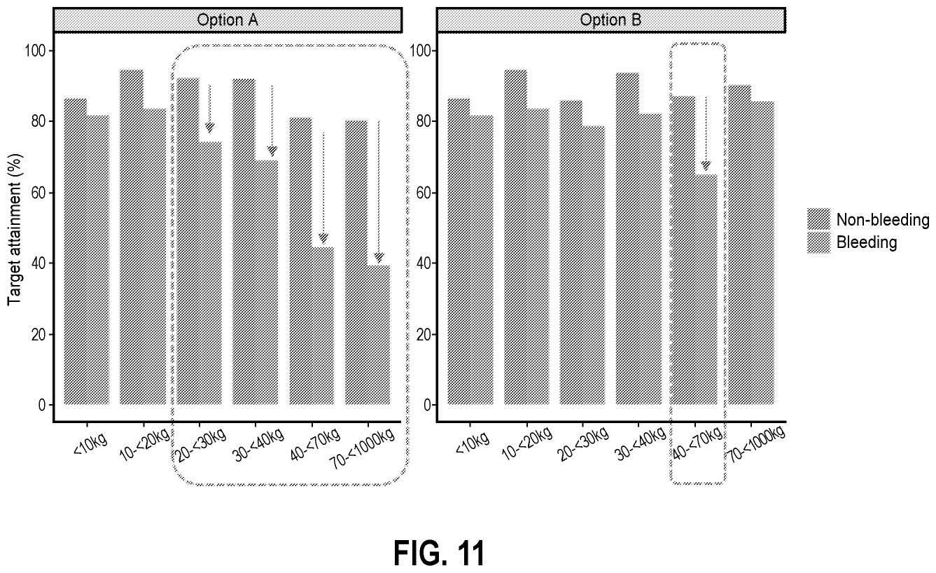

Each dose (mg): (FIG. 11 & FIG. 12). Optimal dosing simulations were conducted based on the published model in Mizuno et al. 2022 with the assumption of similar sC5b-9 distribution as observed in bleeding patients. When patients with active bleeding receive dosing protocol option A (Table 2) for non-bleeding patients, a significantly lower probability of target attainment is predicted for higher body weight cohorts over 20 kg. The simulations in FIG. 12 derived optimized dosing protocols by shortening dosing interval and/or mg dose adjustment for patients >20 kg with active GI bleeding.

Dosing option A and B. (Table 3-A). Two dosing options were proposed for patients with body weight 20-<40 kg, 900 mg IV Q48 h (option A) vs 600 mg IV Q24 h (option B). Considering financial benefits, option A was selected for the optimal dosing proposal for patients with active bleeding. The high clearance in TA-TMA patients with bleeding did not yield financial and pharmacologically beneficial dosing options for patients with patients >40 kg.

### Induction Dose 1

Induction dosing-1 (2-3 weeks). In Applicant's patient cohort, bleeding patients received eculizumab treatment as a median of 13.0 weeks (IQR: 7.3-17.6) whereas non-bleeding patients received 9.8 weeks (IQR 5.9-16.6). Longer treatment was required by a median of three weeks in bleeding patients compared to non-bleeding patients. Induction dosing-1 part therapy may be used in patients who continue to have clinically significant GI bleeding after Loading therapy is completed. The dosing regimen may be administered for three weeks or until active bleeding is controlled (Induction dose-1 therapy course may be shortened or extended based on bleeding status of the patient treated).

Each dose (mg) and dose interval (13 and FIG. 14). SC5b-9 activation is controlled with loading dose, so sC5b-9 is normal when induction dosing-1 is started. Dose given every 7 days is NOT adequate for patients >20 kg due to insufficient C_{trough} target attainment. When bleeding patients receive our proposed loading doses of eculizumab during the induction phase, predicted C_{trough} levels reached over therapeutic levels. The interquartile C_{trough} levels were optimized down to the adequate target level by spacing out by 1-2 days with the same mg dose amount. Further dose adjustment was required for the highest body weight group by adding 1 vial (300 mg) to achieve the interquartile C_{trough} levels above the target level of 100 μg/mL.

Dosing for patients after bleeding is resolved. Patients may proceed to “Induction dosing-2” after GI bleeding is controlled, receiving Loading and Induction-1 dosing parts. Induction 2 may then be followed by the maintenance dosing.

### EXAMPLES

The following non-limiting examples are provided to further illustrate embodiments of the invention disclosed herein. It should be appreciated by those of skill in the art that the techniques disclosed in the examples that follow represent merely exemplary aspects of the disclosure.

### Example 1

Transplant-associated thrombotic microangiopathy (TA-TMA) is a life-threatening complication after hematopoietic stem cell transplantation (HSCT) in pediatric patients and young adults. Patients with high-risk TA-TMA features, including activated terminal complement as measured by elevated blood sC5b-9 and proteinuria, have dismal outcomes with one-year post-transplant overall survival of 16.7%. There are no uniformly agreed on treatment approaches for TA-TMA, but complement dysregulation is an important pathogenic pathway with potential for clinical intervention. Eculizumab, the first available monoclonal antibody against complement C5, showed promising effectiveness for TA-TMA treatment. In HSCT recipients with high-risk complement-mediated TA-TMA, blood soluble terminal complement complex activity (sC5b-9) serves as a surrogate pharmacodynamic biomarker for enhanced C5 production. An eculizumab population PK model after the first dose of therapy allows the prediction of an optimal initial dose as well as the optimal timing for subsequent dose(s) based on the individual pre-treatment sC5b-9 levels and body weight. However, this model (described in Jodele S. et al. Variable Eculizumab Clearance Requires Pharmacodynamic Monitoring to Optimize Therapy for Thrombotic Microangiopathy after Hematopoietic Stem Cell Transplantation. Biology of blood and marrow transplantation: journal of the American Society for Blood and Marrow Transplantation. 2016; 22(2):307-315) only considers elevated sC5b-9 levels at the start of therapy and cannot reflect contextual changes in disease progression and improvement in the subsequent course of therapy.

The significant effect of gastro-intestinal bleeding on eculizumab PK/PD is another factor to consider as part of dose individualization. TA-TMA patients with bleeding have the fastest eculizumab clearance, required the highest number of eculizumab doses (20 vs. 9, p=0.0015), and had lower 1-year survival than those without bleeding (44% vs. 78%, p=0.01). Applicant identified HSCT subjects with TA-TMA, and clinically significant bleeding as an ultra-high-risk group in need of personalized drug dosing to improve survival.

Model-informed precision dosing may be used to improve treatment success by optimizing the drug target exposure. The development of PK/PD model-informed eculizumab precision dosing throughout the therapy promises to improve not only treatment outcome but also cost-effectiveness. Patients with gastrointestinal (GI) bleeding show poor survival even when treated with more frequent dosing. Eculizumab PK/PD were analyzed in 19 bleeding and 38 non-bleeding patients (0.5-29.9 years). A complement activation biomarker (sC5b-9) and bodyweight were identified as significant determinants of eculizumab clearance regardless of bleeding. Eculizumab clearance after the first dose was higher in bleeding patients than in non-bleeding patients (83.8 vs. 61.3 mL/h/70 kg, p=0.07). The high clearance was maintained over treatment doses in bleeding patients, whereas non-bleeding patients showed a time-dependent decrease in clearance. sC5b-9 levels were highest before the first dose and decreased over time regardless of bleeding complications. A Monte Carlo Simulation analysis showed that the current dosing protocols recommended for aHUS had less than 15% probability of eculizumab target concentration attainment of >100 μg/mL in non-bleeding patients.

### Methods

### Study Subjects

A clinical cohort of sixty-four patients with high-risk TA-TMA treated with eculizumab was available for analysis. All study subjects were prospectively and uniformly monitored for TA-TMA and underwent real-time eculizumab PK/PD monitoring. Clinical outcomes of this cohort were recently published in Blood by Jodele et al, Complement blockade for TA-TMA: lessons learned from a large pediatric cohort treated with eculizumab. Jodele S, Dandoy C E, Lane A, Laskin B L, Teusink-Cross A, Myers K C, Wallace G, Nelson A, Bleesing J, Chima R S, Hirsch R, Ryan T D, Benoit S, Mizuno K, Warren M, Davies S M. Blood. 2020 Mar. 26; 135(13):1049-1057. doi: 10.1182/blood.2019004218.PMID: 31932840. Informed consent was obtained from all study subjects participating in the Bone Marrow Transplant Tissue Repository. An institutional review board approved a retrospective analysis of the PK/PD data.

Patient demographics, transplant information, clinical data, and laboratory studies were captured from the electronic medical record and transplant data repository. Red blood cell (RBC) and platelet transfusions were reviewed for each day of eculizumab therapy and documented as ml/kg/day administered for each study subject. Patients with any clinical evidence of lower intestinal bleeding were marked as having clinically significant bleeding and assigned to the bleeding patient group for analysis.

Subjects with incomplete eculizumab concentrations and/or incomplete sC5b-9 data were excluded from the analysis. Eculizumab concentrations ≥1000 μg/mL and eculizumab measurement results when patients were treated with therapeutic plasma exchange (TPE) were excluded from the analysis.

### Eculizumab Treatment and Therapeutic Monitoring

The details of eculizumab dosing, response monitoring methods, and clinical outcomes assessment are described in Jodele S, Dandoy C E, Lane A, et al. Complement blockade for TA-TMA: lessons learned from a large pediatric cohort treated with eculizumab. Blood. 2020; 135(13):1049-1057. Briefly, the first eculizumab dose (mg) was based on bodyweight as suggested in the eculizumab drug label and published data. During the loading and induction phases of therapy, the dosing intervals were adjusted based on eculizumab concentrations and CH50 levels to maintain eculizumab trough concentration in blood at or above 100 μg/mL and trough CH50 levels below 10% of the normal value. Eculizumab loading doses were given at least every 72 hours to subjects with elevated blood sC5b-9 levels at the time of eculizumab therapy start, and loading doses were continued until blood sC5b-9 level normalized (normal <244 ng/mL). Maintenance and taper schedules were only considered after sustaining therapeutic eculizumab concentrations and CH50<10% for at least two consecutive dosing intervals after normalization of sC5b-9. Eculizumab therapy was discontinued after resolution of hematologic TA-TMA and stabilization/improvement in affected organ function when therapeutic eculizumab concentrations and normal sC5b-9 levels were documented for two consecutive doses after starting the taper schedule.

### Eculizumab PK/PD Modeling

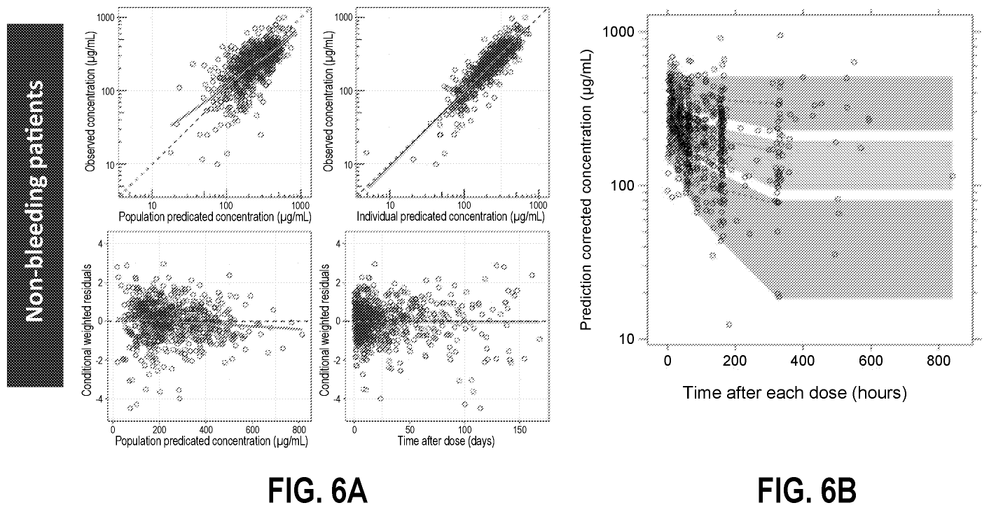

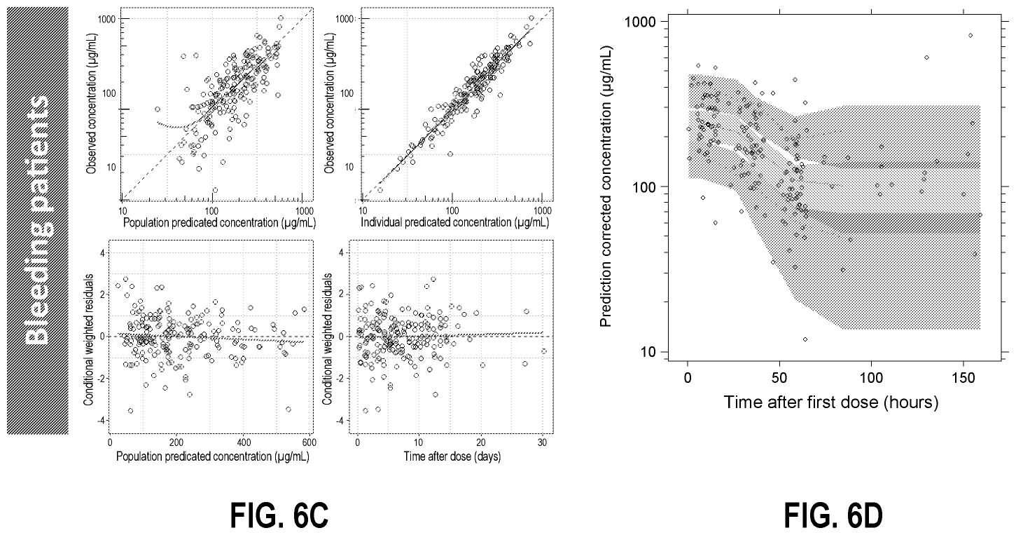

Pharmacokinetic (PK) models for non-bleeding and bleeding TA-TMA patients were developed using nonlinear mixed-effect modeling (NONMEM version 7.4, ICON Development Solutions, Ellicott City, MD, USA) interfaced with Perl-speaks-NONMEM (PsN 4.9.0) and Pirana 2.9.9. According to the criteria of good modeling practice, observations of |conditional weighted residual error |>6 were excluded from the modeling. The effects of potential covariates on eculizumab PK were evaluated using age, sex, body weight, albumin, glomerular filtration rate (GFR), number of eculizumab doses, and sC5b-9 levels. The effect of body size increase on eculizumab clearance was evaluated using allometric scaling to a bodyweight of 70 kg. Inter-occasion variability was also considered. Missing sC5b-9 levels were substituted by an interpolated value between the previous and subsequent values assuming a linear change. The selection of covariates was based on a significant reduction of the objective function value by stepwise forward inclusion (p<0.05), backward elimination (p<0.01), and a graphical evaluation of goodness-of-fit plots. The model evaluation was performed by bootstrap analyses and a prediction-corrected visual predictive check (FIG. 6A-6D).

### Simulation of Eculizumab Concentration-Time Profiles for Non-Bleeding and Bleeding Patients

Eculizumab concentration-time profiles after the first dose were simulated to predict the optimal timing of subsequent doses in representative patients using Edsim++ ver.1.9 based on the developed population PK models for bleeding and non-bleeding patients. Considering the currently recommended starting dose as approved for aHUS (300 mg for <10 kg, 600 mg for 10-<40 kg, and 900 mg for 40 kg and above), representative patients were selected for PK simulations to cover the bodyweight range of the cohorts for young, median and older children/young adults as follows: 8-kg patients receiving 300 mg, 25-kg patients receiving 600 mg, 70-kg patients receiving 900 mg.

### Simulation of Optimal Loading Dose Schedules

Optimal loading dose schedules for TA-TMA non-bleeding patients were explored by evaluating the probability of achieving target trough concentration attainment (PTA %) using a Monte Carlo Simulation (MCS) analysis. PTA % was defined as the proportion of patients achieving the pre-dose eculizumab concentration target of ≥100 μg/mL. A total of 12,000 age-bodyweight-matched subjects was randomly sampled from the CDC-NHANES database using bodyweight cohorts of <10 kg, 10-<20 kg, 20-<30 kg, 30-<40 kg, 40-<70, and 70-<100 kg. Under the assumption of a similar data distribution as observed in the current study, realistic pre-dose sC5b-9 levels were generated using MCS to be matched to the observed sC5b-9 distribution in this study. Five optimal dosing scenarios were selected based on the simulation results for the current recommended loading dose scenarios labeled for aHUS (300 mg for patients <10 kg, 600 mg for patients with 10<−40 kg, and 900 mg for 40-<100 kg). In consideration of the dosage strength of 300 mg per vial, 5 different dosing scenarios were tested as follows: 1; the currently recommended dosing amount for each bodyweight cohort approved for aHUS but with shortening of the dosing interval to 3 days (protocol 1), 2; an increased dosing amount with a 3 day-dosing interval (protocol 2), 3; the currently recommended dosing amount for each bodyweight cohort approved for aHUS but with adjusted dosing interval depending on bodyweight cohort (protocol 3), 4; a combination of increased dosing amount and shortened dosing interval to avoid an extremely high dose (protocol 4), and 5; a mg/kg-based dosing (protocol 5).

### Results

### Study Subjects

Full eculizumab PK/PD data throughout multiple doses of therapy were available in 19 bleeding and 38 non-bleeding patients for the model development. Relevant demographic data for the population PK model development are summarized in Table 1.

Bleeding patients received significantly higher RBC and platelet transfusion than non-bleeding patients during eculizumab therapy time (79-84% of bleeding patients vs. 5% of non-bleeding patients). Pre-treatment sC5b-9 levels in this population were quite variable with a median of 217 ng/mL (107-1641 ng/mL). Bleeding patients had significantly lower albumin and AST values than non-bleeding patients. All other laboratory parameters, including age, bodyweight, pre-dose sC5b-9 levels, were not significantly different between bleeding and non-bleeding patients (Table 1).

### Table 1. Patient Demographics.

GFR, glomerular filtration rate adjusted by 1.73 m2 of body surface area; SCR, serum creatinine, BIL, serum bilirubin, ALB, albumin; AST, aspartate aminotransferase; ALT, alanine aminotransferase, RBC, red blood cell; PLTS, platelet; *1, Total amount, sum of RBC/PLTS transfusion (mg/kg/day) during the first 5 treatment doses; *2, Number of transfusion, sum the RBC/PLTS transfusion days during the first 5 treatment doses

| | Non-bleeding | | Bleeding | | p-value |
|---|---|---|---|---|---|
| Number of patients | 38 | | 19 | | |
| Gender | | | | | |
| Male | 22 | | 14 | | |
| Female | 16 | | 5 | | |
| Race | | | | | |
| Caucasian | 28 | | 16 | | |
| Black/African | 4 | | 3 | | |
| American | | | | | |
| Asian | 3 | | 0 | | |
| Mixed | 3 | | 0 | | |
| Age (years) | 4.9 | (0.5-25.9) | 10.0 | (0.6-29.9) | n.s. |
| Body weight (kg) | 21.3 | (5.0-105) | 29.1 | (5.5-91.6) | n.s. |
| GFR (ml/min/1.73 m²) | 175 | (21.6-501) | 136 | (19.7-894) | n.s. |
| SCR (mg/dL) | 0.4 | (0.2-2.2) | 0.34 | (0.18-1.6) | n.s. |
| BIL (mg/dL) | 0.7 | (1.0-12.5) | 1.2 | (0.2-42.2) | n.s. |
| ALB (mg/dL) | 3.2 | (2.0-4.2) | 2.7 | (2.1-3.4) | <0.01 |
| AST (units/L) | 62 | (13-432) | 38 | (14-135) | <0.05 |
| ALT (units/L) | 39 | (14-643) | 34 | (11-248) | n.s. |
| Pre-dose sC5b9 | 217 | (107-1035) | 339 | (126-1641) | n.s. |
| (ng/mL) | | | | | |
| RBC transfusion | | | | | |
| Number of patients | 5% | (n = 2/38) | 84% | (n = 16/19) | <0.0001 |
| Total amount (ml/kg)^{*1} | 0 | (0-113) | 30 | (0-151) | <0.0001 |
| Number of | 0 | (0-7) | 4 | (0-34) | <0.01 |
| transfusion days^{*2} | | | | | |
| Platelet transfusion | | | | | |
| Number of patients | 5% | (n = 2/38) | 79% | (n = 15/19) | <0.0001 |
| Total amount (ml/kg)^{*1} | 0 | (0-328) | 48 | (0-220) | <0.0001 |
| Number of | 0 | (0-23) | 7 | (0-65) | <0.0001 |
| transfusion days^{*2} | | | | | |
| First loading dose | | | | | |
| 300 mg | 6 | | 4 | | |
| 600 mg | 23 | | 6 | | |
| 900 mg | 9 | | 9 | | |

TABLE 2
Population PK parameter estimates

| | Non-bleeding | Bleeding |
|---|---|---|
| CL_{tot,pop} (m/h/70 kg) | | 87.2 (60.0, 27.2 fixed) |
| (CL_{NL,pop}. CL_{L,pop}) | | |
| 1^{st} dose | 58.5 (31.3, 27.2) | |
| 2^{nd}-< 5^{th} dose | 44.7 (17.5, 27.2) | |
| ≥5^{th} dose | 41.6 (14.4, 27.2) | |
| Vdₚₒₚ (L/70 kg) | | 4.4 |
| 1^{st} dose | 5.5 | |
| 2^{nd}-<5^{th} dose | 6.5 | |
| ≥5^{th} dose | 8.0 | |
| Exponent for pre- | 0.53 | 0.52 |
| dose sC5b-9 | | |
| Exponent for | 0.97 for CL | 1.03 for CL |
| body weight | 0.63 for Vd | 0.74 for Vd |
| Inter-individual | 44.7% for CL | 31.6% for CL |
| variability | 33.6% for Vd. | 34.5% for Vd |
| Residual proportional | 0.11 | 0.084 |
| variability | | |

Final model for non-bleeding patients is as follows:

CL=CL_{L}+CL_{NL},CL_{L}=CL_{L,pop}×(WT/70)^{0.97},CL_{NL}=CL_{NL,pop}×(sC5b-9/244)^{0.53}×(WT/70)^{0.97},

Vd=Vdₚₒₚ×(WT/70)^{0.63}.
Final model for bleeding patients is as follows:

CL=CL_{L}+CL_{NL},CL_{L}=CL_{L,pop}×(WT/70)^{1.03},CL_{NL}=CL_{NL,pop}×(sC5b-9/244)^{0.52}×(WT/70)^{1.03},

Vd=Vdₚₒₚ×(WT/70)^{0.74}.
where CL_{NL,pop} is the eculizumab population mean nonlinear clearance for a 70 kg-patient, CL_{L,pop}, is the eculizumab population mean linear clearance for a 70 kg-patient, CL_{tot,pop} the population mean total clearance defined as sum of CL_{NL,pop} and CL_{L,pop}, Vdₚₒₚ, is the population mean volume of distribution for a 70 kg-patient, and WT is actual body weight (kg).

### Eculizumab PK/PD Differences Between Non-Bleeding and Bleeding Patients

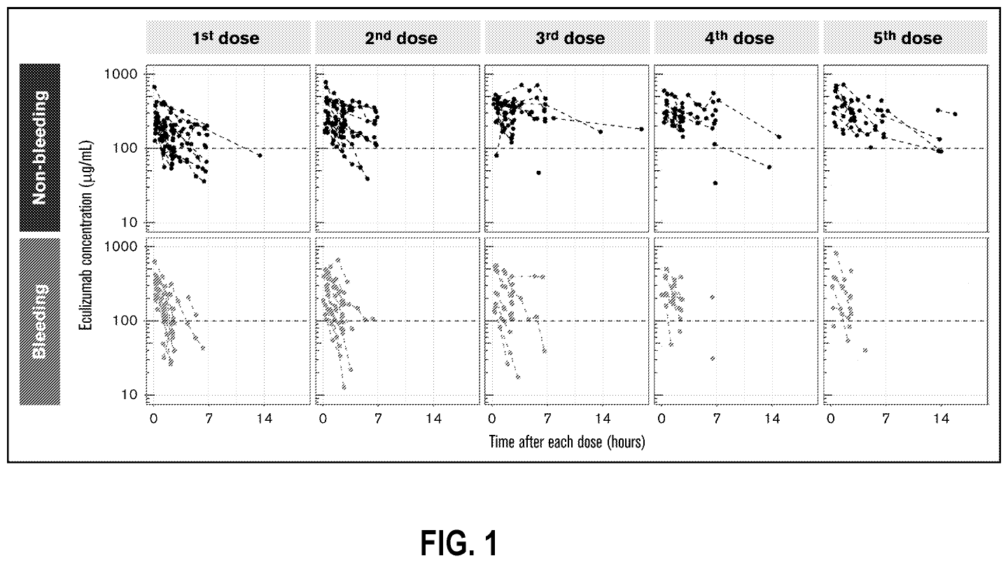

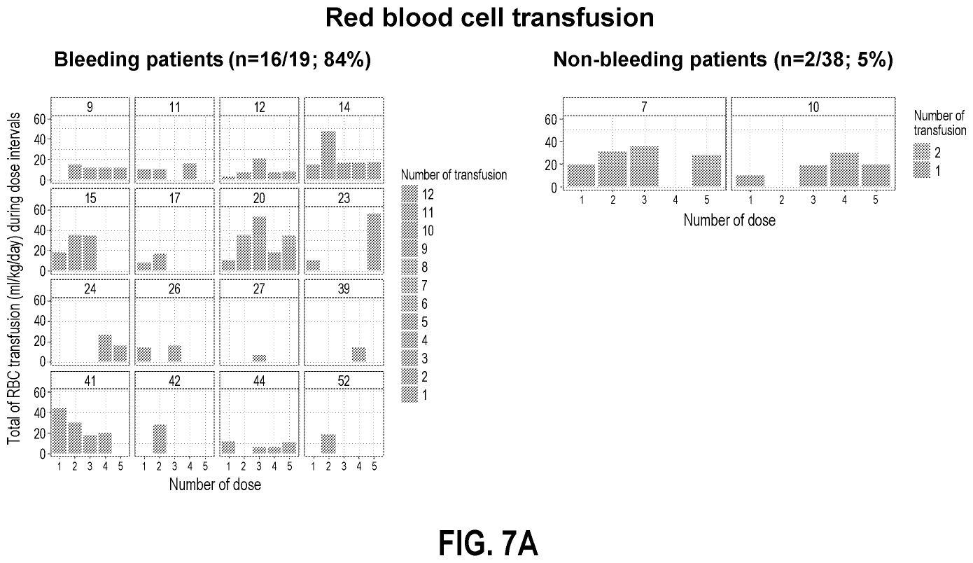

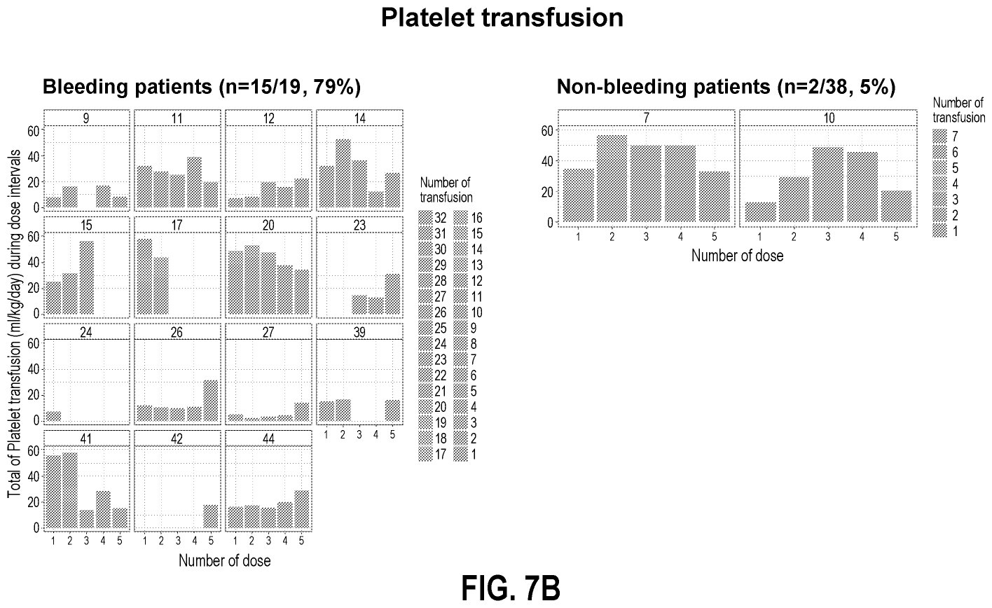

Eculizumab concentration-time profile changes were visualized during the first 5 doses of therapy in non-bleeding and bleeding patients (FIG. 1). In bleeding patients, eculizumab concentrations fell below the target level more rapidly across treatment doses compared to non-bleeding patients. Drug elimination gradually decreased over the doses of therapy in non-bleeding patients. By contrast, bleeding patients maintained high drug elimination across all five doses. Bleeding patients received higher RBC and platelet transfusion across all first 5 doses of therapy compared to non-bleeding patients (FIG. 7A, 7B).

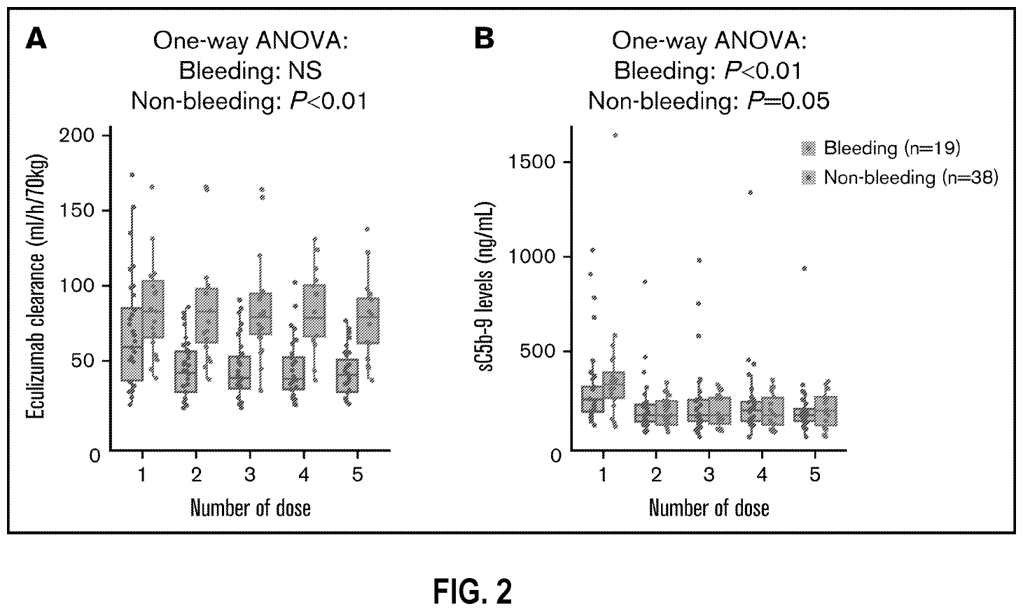

Eculizumab PK/PD differences between non-bleeding and bleeding patients were also evaluated by using individual eculizumab clearance estimates and observed pre-dose sC5b-9 levels. Median eculizumab clearance after the first dose tended to be higher in bleeding patients than in non-bleeding patients (83.8 vs. 61.3 mL/h/70 kg, p=0.07). It is important to note that high clearance was maintained over time in bleeding patients, whereas non-bleeding patients showed a decrease in clearance over time. Pre-dose sC5b-9 levels were highest during the first week of therapy and decreased over time regardless of bleeding events (FIG. 2, B). Predose sC5b-9 levels tended to be higher in bleeding patients during the first week, followed by suppression to comparable levels as in non-bleeding patients during subsequent therapy.

### Eculizumab Population PK Modeling

Separate population PK models were developed for non-bleeding and bleeding patients. A one-compartment model best described the data. Eculizumab clearance (CL) was described by a nonspecific linear clearance (CL_{L}) component representing the neonatal Fc receptor-mediated clearance and a non-linear clearance (CL_{N}) component for target-mediated clearance.²²⁻²⁴ In bleeding patients, CL_{L} and CL_{NL} could not be estimated independently due to no observed eculizumab clearance changes over time. Considering very high total clearance observed in bleeding patients, the effect of CL_{L} difference between bleeding and non-bleeding patients could be ignored. Therefore, CL_{L} estimated in non-bleeding patients was used for the PK modeling for bleeding patients.

Regardless of bleeding events, sC5b-9 level and bodyweight were significant covariates predictive of eculizumab clearance with comparable power exponent estimates. Non-bleeding patients showed a mean eculizumab clearance estimate after the first dose of 58.5 mL/h (CL_{N}; 31.3 mL/h, CL_{L}; 27.2 mL/h) in a 70-kg patient and at the upper normal range of the sC5b-9 level of 244 ng/mL. These estimates are comparable to Applicant's previously published model, which derived a mean eculizumab clearance of 66.0 mL/h in a 70-kg patient with an sC5b-9 level of 244 ng/mL. The eculizumab non-linear time-dependent decrease in clearance was a function of the number of doses in addition to sC5b-9 changes. The mean volume of distribution after the first dose for a 70-kg patient was 5.5 L. The volume of distribution increased up to 63% over the doses of therapy.

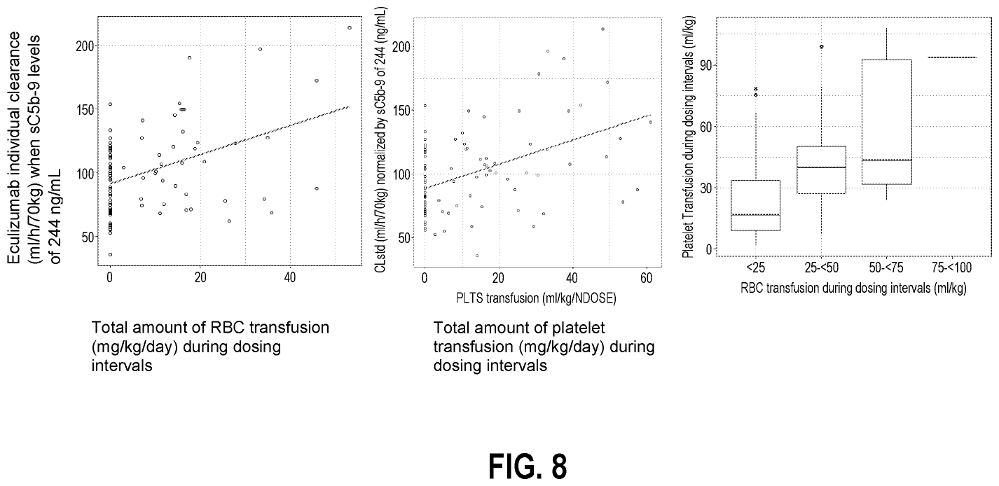

Bleeding patients had a mean eculizumab clearance of 87.2 mL/h (CL_{N}; 60.0 L/h, CL_{L}; 27.2 L/h) after the first dose in a 70-kg patient at an sC5b-9 level of 244 ng/mL, which was 50% higher than the mean clearance estimate in non-bleeding patients. The eculizumab non-linear clearance remained constant over the multiple doses of therapy when bodyweight and sC5b-9 levels did not change. Bleeding patients had a volume of distribution of 4.4 L (normalized to a 70-kg patient) across different dosing intervals, which was 20% lower than the volume of distribution after the first dose in non-bleeding patients. The amount of RBC correlated with the amount of platelet transfusion. Patients who received RBC and/or platelet transfusion were likely to have higher eculizumab clearance (R²=0.13 for RBC and R²=0.20 for platelet transfusion, FIG. 8). However, this effect was not significant and was not retained in the final model.

### Eculizumab PK Simulations

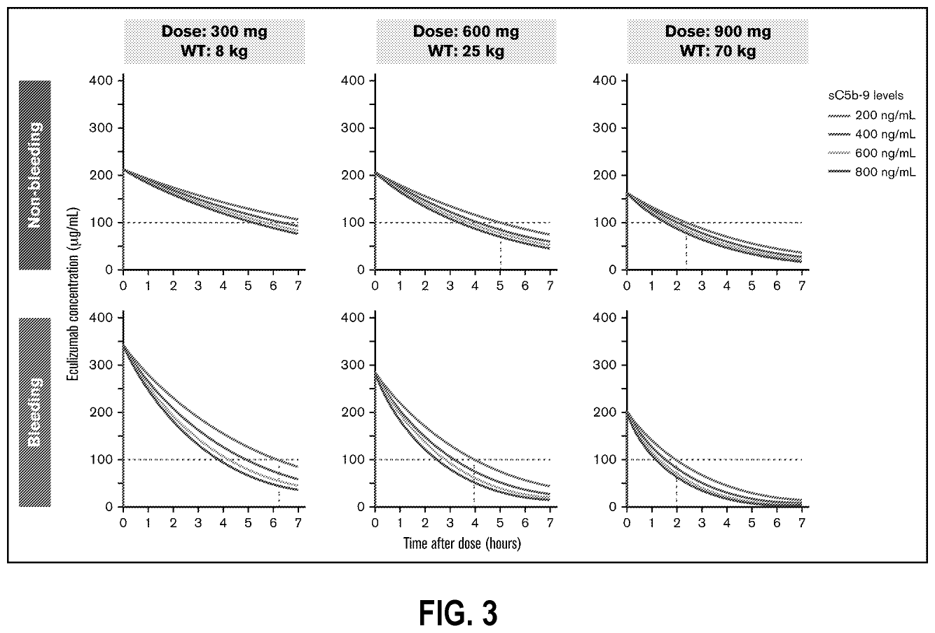

Previously reported eculizumab concentration-time simulations covering the 7 days after the first dose of therapy were expanded based on the newly developed PK model for patients with bleeding complications (FIG. 3). The simulations predicted the timing of the next dose required to maintain eculizumab target concentrations at ≥100 μg/mL in representative cases. FIG. 3 provides a summary of the simulated eculizumab concentration-time profiles for the dosing scenarios in 8-kg patients receiving 300 mg, 25-kg patients receiving 600 mg, and 70-kg patients receiving 900 mg.

In 25-kg non-bleeding patients receiving 600 mg eculizumab with a pre-dose sC5b-9 level of 400 ng/mL, the mean eculizumab concentration was predicted to decline below 100 μg/mL at around 4 days after the first dose. The mean eculizumab concentration was maintained above the target for 5 days when a lower pre-treatment sC5b-9 level of 200 ng/mL would be present. The 8-kg non-bleeding patients receiving 300 mg eculizumab were predicted to maintain eculizumab concentration above the target for a period ranging from 5 to 7 days depending on pre-dose sC5b-9 levels. This target attainment period was longer than what was observed in 25-kg patients receiving 600 mg eculizumab. In contrast, 70-kg non-bleeding patients receiving 900 mg were predicted to have their eculizumab concentration fall below target within 3 days after the first dose. In bleeding patients, eculizumab concentration was predicted to fall below the target approximately 0.5-1 day earlier than what was predicted in non-bleeding patients.

### Simulation of Optimal Dosing Schedules

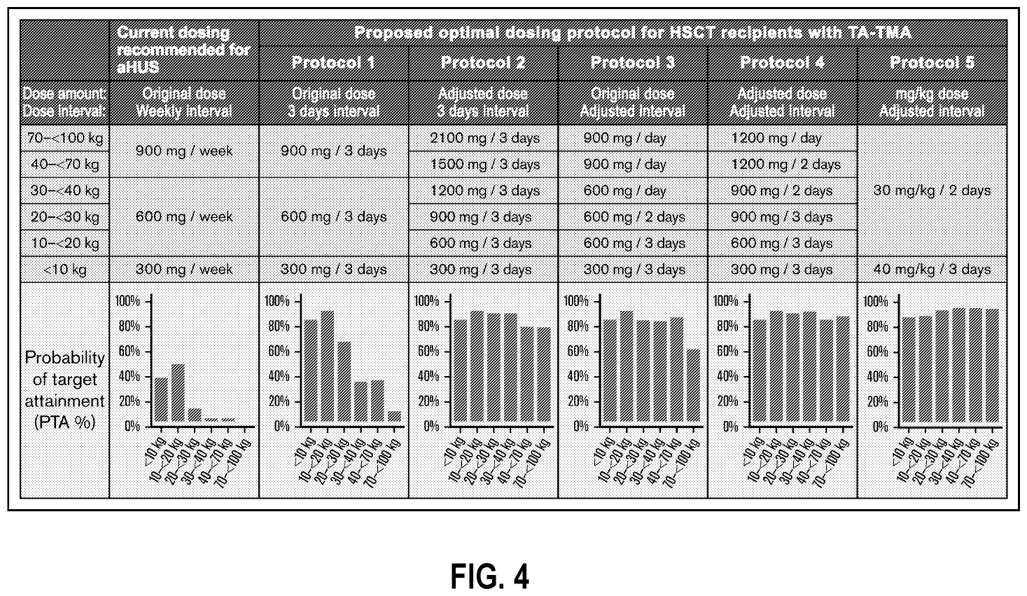

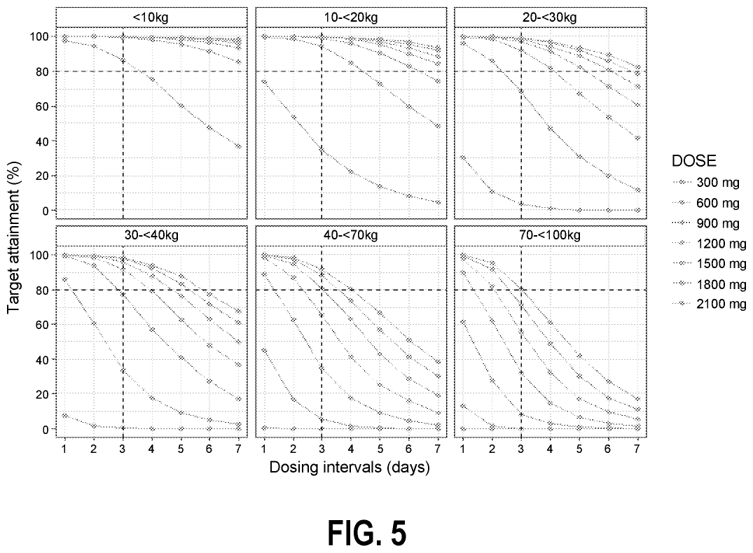

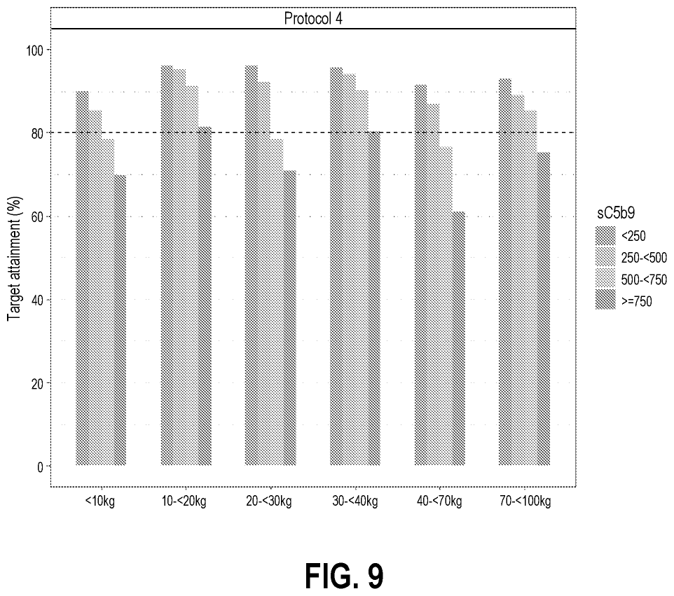

The currently recommended weekly induction dosing approved for aHUS resulted in a maximal probability of target attainment (PTA %) of 50% in non-bleeding patients. Lower PTA % was predicted for patients with higher body weights (≥20 kg) compared to those <20 kg. When intensifying the eculizumab dosing by using the same amount of drug (mg) per dose but given every 3 days (Protocol 1), a PTA % of at least 80% was achieved in subjects <20 kg. PTA % was below 50% in patients ≥20 kg. Therefore, Protocol 2 evaluated an optimal dose, especially for patients ≥20 kg with a fixed 3 day-dosing interval to reach at least 80% PTA by subdividing body-weight cohorts. With 3 day-dosing intervals, the optimal doses were 900 mg for patients weighing 20-<30 kg, 1200 mg for 30-<40 kg, 1500 mg for 40-<70 kg. In patients ≥70 kg, the optimal dose was predicted to be 2100 mg, which is significantly more than the currently recommended maximum induction dose of 900 mg. Protocol 3 evaluated the best dosing interval to reach 80% PTA when the recommended aHUS dose amount (mg) was selected for each body-weight cohort. The predicted optimal interval was 3 days for patients weighing <20 kg, 2 days for 20-<30 kg, and 1 day for ≥30 kg. The currently recommended 900 mg dose resulted in only up to 60% PTA in patients weighing 70 to 100 kg even if they were administered the dose on a daily basis. Protocol 4 evaluated the optimal dosing protocol by combining increased dosing amount (mg) and dose intensification. The predicted optimal dose protocol was 900 mg every 3 days for patients weighing 20-<30 kg, and 900 mg every 2 days for 30-<40 kg, 1200 mg every 2 days for 40-<70 kg, and 1200 mg every 2 days for 40-<70 kg and 1200 mg daily for ≥70 kg. Lastly, the optimal mg/kg-based dose to reach 80% PTA is proposed in Protocol 5 as an option. The optimal dose for patients weighing less than 10 kg was 40 mg/kg every 3 days, and 30 mg/kg every 2 days for patients of 10 kg or more. A summary of the dosing protocols achieving various PTA % targets can be found in FIG. 4. PTA % for the various dosing schedules with dosing amounts ranging from 300 to 2100 mg and dosing intervals ranging from 1 to 7 days are described. FIG. 5 shows that a PTA % close to 100% could be achieved by increasing the dose by 300 mg (one vial) in each bodyweight cohort of protocol 4. The effect of pre-dose sC5b-9 levels on PTA % is summarized in FIG. 9. The PTA % predictions for dosing protocol 4 were stratified by sC5b-9 cohorts of <250, 250-<500, 500-<750 and >750 ng/mL. PTA % declined along with increases in sC5b-9 levels. PTA % was predicted to be lower than 80% in patients with high sC5b-9 levels of ≥500 ng/mL.

### DISCUSSION

Sustained excessive complement activation is damaging to the endothelium and may result in multi-organ injury and death. Applicant's prior clinical observations clearly demonstrate that prompt complement blockade is needed to improve clinical outcomes in HSCT recipients with high-risk TA-TMA. The most precise drug dosing is required during the loading and induction phases of the therapy when complement and TA-TMA activity is the highest. This newly developed eculizumab loading, induction, and maintenance dosing algorithm confirms that the dosing regimen currently approved for patients with aHUS is not suitable for HSCT recipients with TA-TMA due to significantly low target attainment. These results strongly suggest that eculizumab dosing for HSCT patients with TA-TMA needs to be optimized.

Disclosed are population PK models using an enriched eculizumab PK/PD dataset collected throughout multiple eculizumab treatment doses in the largest TA-TMA cohort of HSCT recipients. The model updates add several features for future clinical applications to previously published models. The updated model can be used for the precision dosing of eculizumab, not only for the first dose, but also for subsequent doses. The enriched eculizumab PK/PD dataset allowed for development of models considering the mechanistic operational concepts of the elimination pathways of the monoclonal antibodies: i.e., Brambell receptor-mediated elimination and target mediated-elimination pathways. Currently approved aHUS dosing guidelines use the same induction dose in patients with body weights ranging from 10 to 40 kg. The simulations in HSCT patients showed that the target attainment for patients with ≥20 kg was significantly lower compared to those with <20 kg when they were treated with the same dosing protocol. These observations suggest that further subdivided bodyweight groups are beneficial in HSCT patients to avoid below target concentrations in higher bodyweight patients.

Bleeding patients have much higher eculizumab drug clearance. There is a great benefit to optimize the eculizumab dose depending on the bleeding complications, as preliminary analysis showed that bleeding patients show different pharmacological and disease characteristics than what is observed in non-bleeding patients.

This newly developed model is also applicable for eculizumab dose optimization during the maintenance phase. Eculizumab predicted clearance during the maintenance phase was 27.2 mL/h when sC5b-9 levels are in the normal range, which was still 23% higher than the reported clearance for aHUS. This suggests that more frequent dosing may be required in TA-TMA patients than the currently recommended dosing intervals approved for aHUS suggest for the maintenance phase.

Bleeding patients had significantly lower survival as compared to non-bleeding patients (44% vs. 78%). Applicant compared eculizumab PK/PD in bleeding patients to those in patients without bleeding. RBC and platelet transfusion requirements were used as surrogate markers for microangiopathic-hemolytic activity and blood loss. Impressively, only 5% of non-bleeding patients required transfusions after initiating eculizumab therapy indicating good control of hemolysis and platelet consumption, while 84% of bleeding patients were transfusion dependent. Consistent with higher transfusion requirements across treatment dosing intervals observed in bleeding patients, eculizumab clearance remained high over the doses of therapy, whereas clearance decreased over time in non-bleeding patients. Interestingly, there was no significant difference in time-dependent sC5b-9 reduction between bleeding and non-bleeding patients. This can be explained by the more frequent dosing applied in the bleeding patients as the treatment study used real-time eculizumab concentrations and CH50 monitoring for dose adjustments. These results suggest that a model-informed precision dosing strategy with consideration of bleeding may reduce sC5b-9 quickly and effectively by achieving eculizumab target concentrations. Adequate complement blockade in patients with intestinal bleeding may still not be enough to improve survival as these patients often have other transplant-related complications like graft versus host disease (GVHD) or infections. An early intervention preserving vascular endothelial health and prompt complement activation control along with effective GVHD prophylaxis or therapy and infection control is likely required to further improve clinical outcomes.

Applicant sought to elucidate the mechanism of high eculizumab clearance in bleeding patients. In this study, pre-dose sC5b-9 level and patient weight remained significant covariates predictive of high drug clearance without any new covariates being identified. One possibility was that patients with severe blood loss had high eculizumab clearance due to drug loss from the body. Our covariate analysis suggested that red blood cell transfusion as a surrogate marker for bleeding severity in bleeding patients could partly explain the high clearance, although it was not retained in the final model. Another potential mechanism is sustained excessive C5 generation from injured bowel tissue providing large number of targets for eculizumab to bind to. PK/PD analysis showed that higher pretreatment sC5b-9 at the start of therapy reflected high drug clearance; however, eculizumab clearance in bleeding patients remained high even after sC5b-9 value normalized, potentially indicating ongoing C5 generation that continues to require eculizumab for blockade to maintain normal sC5b-9 level. The lower albumin levels in bleeding patients could be partly involved in the high clearance as reported for other monoclonal antibodies (mAbs) such as infliximab and anti-PD-L1 antibody. In fact, Applicant's study showed that the baseline albumin levels in bleeding patients were significantly lower than in non-bleeding patients. However, the low albumin may cause increased protein turnover, resulting in facilitating degradation of IgG, including mAb, and an increase in mAb neonatal Fc receptor-mediated clearance. In this study, there was no significant effect of albumin on eculizumab disposition, possibly because bleeding patients were receiving total parenteral nutrition containing albumin, which could have masked the effect. This suggests that high drug clearance is likely multi-factorial in bleeding patients and such patients require personalized PK/PD-based dosing of eculizumab.

In summary, Applicant's study shows that HSCT recipients require a dedicated drug dosing schedule suitable for this population. Several dosing strategies were identified that can be incorporated into clinical care. Prior to Applicant's invention, “fixed”-dose or “blanket” dosing regimens could be derived for eculizumab dosing in non-bleeding HSCT recipients, but were not satisfactory for those with clinically significant bleeding. While Applicant showed that TA-TMA patients with bleeding benefit from personalized dose adjustments using continuous PK/PD dose modification in order to provide adequate eculizumab exposures based on disease activity, the “fixed” dosing table can be specifically made for bleeding patients to be used by care givers who do not have access to personalized dosing tool. Bleeding patients need more intense loading and induction therapy course, likely due to sustained C5 generation from the injured bowel and some drug loss due to bleeding. Such personalized dosing tools may be used in clinical practice for bleeding patients to further improve post-transplant outcomes.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A method of treating an individual with transplant-associated thrombotic microangiopathy (TA-TMA) and active gastrointestinal bleeding, comprising administering eculizumab, or antigen binding fragment thereof, to said individual wherein said eculizumab or antigen binding fragment thereof is administered
a loading dose and an induction dose,
said loading dose comprising
1200 milligrams every 24 hours for four doses, followed by 1500 milligrams every 48 hours for five doses, wherein said individual is 70 kilograms or more, or 900 milligrams every 24 hours for four doses followed by 900 milligrams every 48 hours for five doses, wherein said individual has a weight of 40 kilograms to less than 70 kilograms, or 900 milligrams every 48 hours for two doses followed by every 72 hours for three doses, wherein said individual has a weight of 30 kilograms to less than 40 kilograms, or 900 milligrams every 48 hours for two doses followed by 900 milligrams every 72 hours for three doses, wherein said individual has a weight of 20 kilograms to less than 30 kilograms, or 600 milligrams every 72 hours for five doses, wherein said individual has a weight of 10 kilograms to less than 20 kilograms or 300 milligrams every 72 hours for five doses, wherein said individual has a weight of less than 10 kilograms;
said induction dose comprising
1500 milligrams every 48 hours, wherein said individual has a weight of 70 kilograms or more, or 900 milligrams every 48 hours, wherein said individual has a weight of 40 kilograms to less than 70 kilograms, or 900 milligrams every 72 hours, wherein said individual has a weight of 30 kilograms to less than 40 kilograms, or 900 milligrams every 96 hours, wherein said individual has a weight of 20 kilograms to less than 30 kilograms, or 600 milligrams every 96 hours, wherein said individual has a weight of 10 kilograms to less than 20 kilograms, or 300 milligrams every 96 hours, wherein said individual has a weight of less than 10 kilograms.

2. A method of treating an individual with transplant-associated thrombotic microangiopathy (TA-TMA) and resolved gastrointestinal bleeding, comprising administering eculizumab, or antigen binding fragment thereof, to said individual wherein said eculizumab or antigen binding fragment thereof is administered
an induction dose and a maintenance dose,
said induction dose comprising
1500 milligrams twice weekly every three to four days for four weeks, wherein said individual has a weight of 70 kilograms or more, or 1500 milligrams weekly for four weeks, wherein said individual has a weight of 40 kilograms to less than 70 kilograms, or 1200 milligrams weekly for four weeks, wherein said individual has a weight of 30 kilograms to less than 40 kilograms, or 900 milligrams weekly for four weeks, wherein said individual has a weight of 20 kilograms to less than 30 kilograms, or 600 milligrams weekly for four weeks, wherein said individual has a weight of 10 kilograms to less than 20 kilograms, or 300 milligrams weekly for four weeks, wherein said individual has a weight of less than 10 kilograms;
said maintenance dose comprising
1200 milligrams twice weekly every three to four days for four to five weeks, wherein said individual is 70 kilograms or more, or 1200 milligrams weekly for four to five weeks, wherein said individual has a weight of 40 kilograms to less than 70 kilograms, or 900 milligrams weekly for four to five weeks, wherein said individual has a weight of 30 kilograms to less than 40 kilograms, or 600 milligrams weekly for four to five weeks, wherein said individual has a weight of 20 kilograms to less than 30 kilograms, or 600 mg weekly for four to five weeks, wherein said individual has a weight of 10 kilograms to less than 20 kilograms, or 300 milligrams every two weeks for four to five weeks, wherein said individual has a weight of less than 10 kilograms.

3. (canceled)

4. (canceled)

5. (canceled)

6. (canceled)

7. The method of claim 1, where said TA-TMA is high-risk TMA with MODS.

8. The method of claim 1, wherein said TA-TMA is characterized by a sC5b-9 level that is at least twice the measured baseline level in said individual.

9. The method of claim 1, wherein said TA-TMA is characterized by a sC5b-9 level that is greater than 244 nanograms per milliliter.

10. The method of claim 1, wherein said administration is initiated at the time of HSCT-TMA diagnosis in said individual.

11. The method of claim 1, wherein said administration is intravenous administration.

12. The method of claim 1, wherein said administration is carried out until a hematological TMA response selected from one or more of normalization of LDH, resolution of the need for red blood cell (RBC) and platelet transfusions, and disappearance of schistocytes is achieved.

13. The method of claim 1, wherein said method comprises an induction dose and said induction dose is carried out until a hematological TMA response of normalization of LDH, resolution of the need for red blood cell (RBC) and platelet transfusions, and disappearance of schistocytes is achieved.

14. The method of claim 1, wherein said method comprises an induction dose and wherein said induction dose is carried out until a normalized sC5b-9 is achieved, said normalized sC5b-9 being one or both of an sC5b-9 level of less than 244 ng/mL or an sC5b-9 level that is substantially at baseline (pre-transplant), or until an elevated CH50 (total blood complement) level is normalized.

15. The method of claim 1, wherein said individual has clinically significant bleeding.

16. The method of claim 1, wherein said individual does not have clinically significant bleeding (non-bleeding status individual).

17. The method of claim 1, wherein said individual is an infant.

18. The method of claim 1, wherein said individual is pre-pubescent.

19. The method of claim 1, wherein said individual is an adult.

20. A precision dosing tool for determining a course of treatment in an individual diagnosed with TA-TMA, comprising detecting one or more patient-specific variables, and determining a therapeutically effective amount of an eculizumab therapy, wherein said determining employs an algorithm selected from
CL=CL_{L}+CL_{NL},CL_{L}=CL_{L,pop}×(WT/70)^{0.97},CL_{NL}=CL_{NL,pop}×(sC5b-9/244)^{0.53}×(WT/70)^{0.97},
Vd=Vdₚₒₚ×(WT/70)^{0.63}(for non-bleeding patients); and
CL=CL_{L}+CL_{NL},CL_{L}=CL_{L,pop}×(WT/70)^{1.03},CL_{NL}=CL_{NL,pop}×(sC5b-9/244)^{0.52}×(WT/70)^{1.03},Vd=Vdₚₒₚ×(WT/70)^{0.74} (for bleeding patients);
wherein CL_{NL},pop is the eculizumab population mean nonlinear clearance for a 70 kg-patient which represents a target-mediated component of clearance, CL_{L,pop}, is the eculizumab population mean linear clearance for a 70 kg-patient which represents a nonspecific component of clearance mediated by the neonatal Fc receptor, CL_{L,pop}, is the eculizumab population mean linear clearance for a 70 kg-patient, CL_{tot,pop} is the population mean total clearance defined as sum of CL_{NL,pop} and CL_{L,pop}, Vdₚₒₚ, is the population mean volume of distribution for a 70 kg-patient, and WT is actual body weight (kg); and
administering a therapeutically effective amount of eculizumab to said individual according to said algorithm.

21. The precision dosing tool of claim 20, wherein said determining is carried out by a computerized device.
